(19) United States
(12) Patent Application Publication
(10) Pub. No.: US 2015/0038688 A1
(43) Pub. Date: Feb. 5, 2015

(54) USE OF METALLOPORPHYRINS AND SALEN COMPLEXES FOR THE CATALYTIC OXIDATION OF ORGANIC COMPOUNDS

(71) Applicant: Thinq Pharma, Natick, MA (US)

(72) Inventor: Mukund CHORGHADE, Natick, MA (US)

(21) Appl. No.: 14/460,781

(22) Filed: Aug. 15, 2014

Related U.S. Application Data
(63) Continuation of application No. PCT/US13/26731, filed on Feb. 19, 2013.
(60) Provisional application No. 61/600,487, filed on Feb. 17, 2012.

(51) Int. Cl. C07C 37/00; C08B 15/00

(52) U.S. Cl. CPC C07C 37/00; C08B 15/00
USPC 534/588; 536/56; 568/800

(57) ABSTRACT

A method of decomposing an organic substrate includes identifying an organic substrate or its constituents having one or more desired or undesired properties; and contacting the organic substrate with an oxidizing agent and a catalyst selected from the group consisting of sterically hindered and electronically activated metallotetraphenylporphyrins, metallophthalocyanines and metallosalen complexes in an aqueous or aqueous-organic solution to produce a treated composition comprising one or more degradation products, wherein the degradation products have one or more desired properties and/or lack the undesired properties of the organic substrate.

### RELATED APPLICATIONS

This application is a continuation of PCT International Application No. PCT/US2013/026731 filed Feb. 19, 2013, entitled “Use of Metalloporphyrins and Salen Complexes for the Catalytic Oxidation of Organic Compounds”, which claims the benefit of priority to U.S. Application No. 61/600,487, filed on Feb. 17, 2012 and entitled “Use of Metalloporphyrins and Salen Complexes for the Catalytic Oxidation of Organic Compounds”, which are incorporated herein in their entirety by reference.

### INCORPORATION BY REFERENCE

All patents, patent applications and publications cited herein are hereby incorporated by reference in their entirety in order to more fully describe the state of the art as known to those skilled therein as of the date of the invention described herein.

### TECHNICAL FIELD

This technology relates generally to novel catalysts for the catalytic oxidation of organic substrates such as polymers and small molecules for pharmaceutical or agrochemical use. It also relates to new and improved methods for synthesizing porphyrin and other azamacrocycle catalysts.

### BACKGROUND

Most biological oxidations involve primary catalysis provided by the cytochrome P-450 mono-oxygenase enzymes. All heme proteins that are activated by hydrogen peroxide, including catalases, peroxidases and ligninases function via a two electron oxidation of the ferric resting state to an oxoferryl porphyrin cation radical (I).

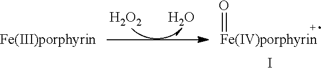

While this oxidation state has yet to be characterized for the cytochromes P-450, most of their reactions and those of the biomimetic analogs can be accounted for by oxygen transfer from (I) to a variety of substrates to give characteristic reactions such as hydroxylation, epoxidation and heteroatom oxidation. Other products resulting from hydroxyl and hydroperoxyl radicals have also been detected.

The first synthetic metalloporphyrins studied were found to be unstable. But improvements in molecular stability and increases in the turnover of catalytic reactions have been obtained with the introduction of additional atoms into the synthetic metalloporphyrin molecules. The work of Dolphin and others has shown that addition of halogen atoms onto the aryl groups and the pyrrolic positions of meso-tetraarylporphyrins makes intermediate oxo-porphyrin complexes more electron deficient and more sterically protected and thus provides for more effective oxidation catalysis. See, for example, Traylor, P. S.; Dolphin. D.; Traylor. T. G. J. Chem. Soc. Chem. Commun. 1984, 279 and M. S. Chorghade*, D. H. Dolphin*, D. Dupre, D. R. Hill, E. C. Lee and T. P. Wijesekara, “Improved Protocols for the Synthesis and Halogenation of Sterically Hindered Metalloporphyrins”, Synthesis, 1996, 1320.

Efficient methods for the catalytic oxidation of organic compounds are desired.

### SUMMARY

In one aspect, a method of decomposing an organic substrate includes identifying an organic substrate having one or more undesired properties; and contacting the organic substrate with an oxidizing agent and a catalyst selected from the group consisting of sterically hindered and electronically activated metallotetra aryl porphyrins, metallophthalocyanines and metallosalen complexes in an aqueous solution to produce a treated composition comprising one or more degradation products, wherein the degradation products have one or more desired properties and/or lack the undesired properties of the organic substrate.

In one or more embodiments, the organic substrate is toxic and the degradation products are less toxic than the organic substrate.

In one or more embodiments, the degradation products have increased water solubility relative to the organic substrate.

In one or more embodiments, the organic substrate is a polymer and the degradation polymer is one or more of monomers or oligomers.

In one or more embodiment, the organic substrate includes molecules having unsaturated moieties such as alkenes C═C, alkynes, or azo derivatives, and preferably in conjugation with at least one other unsaturated moiety.

In one or more embodiments, the polymer comprises lignin and, for example, the decomposition product comprises a phenol.

In one or more embodiments, the substrate is an organic dye, and for example, the decomposition product is a water soluble, colorless reaction product.

In one or more embodiments, the catalyst is a meso-tetraphenyl porphyrin.

In one or more embodiments, the catalyst is phthalocyanine.

In one or more embodiments, the meso-tetraphenylporphyrin catalyst comprises at least one halide substitution on the phenyl groups of meso-tetraphenylporphyrins or on the β-pyrrolic positions of the porphyrin.

In one or more embodiments, the phthalocyanine comprises at least one halide substitution on the benzo groups of the phthalocyanine.

In one or more embodiments, the catalyst is a compound

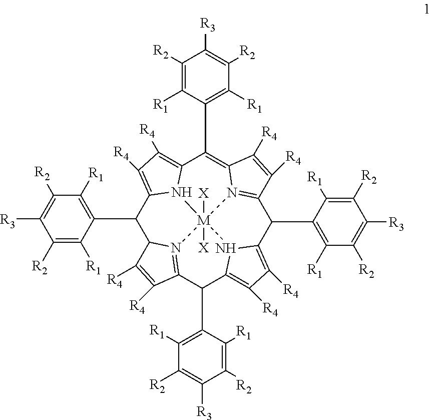

wherein R¹ is the same or different and is selected from the group consisting of Cl, Br, CH₃, SO₃⁻, CN, [N(R′)₃]⁻, COOR′, —OCONR′₂, —OMOM, CON—R′, CONR′₂, CH═NR′, SO₂NR′₂, SO₂R, CF and NO₂,
R², R³ or R⁴ are the same or different and are selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,
R′ is H or a C1-C6 alkyl,
M is a transition metal, such as Fe, Zn, Co, Ni, Cu, Mn, Rh, Mg, Ru, Pt, and Pd., and
and optionally wherein one or more axial ligands X selected from the group halogens (F, Cl, Br), OH, OCl, CO, [N(R′)₃]⁺, substituted or unsubstituted pyrimidine or imidazole bases is included and/or a counter ion is included to maintain charge neutrality.

In one or more embodiments, the catalyst is a compound

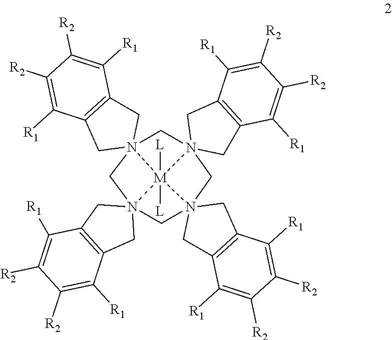

wherein R¹ is the same or different and is selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,

R² is the same or different and is selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,

wherein R′ is H or a C1-C6 alkyl,

M is a transition metal, such as Fe, Zn, Co, Ni, Cu, Mn, Rh, Mg, Ru, Pt, and Pd,

and optionally wherein one or more axial ligands X selected from the group halogens (F, Cl, Br), OH, OCl, CO, [N(R′)₃]⁺, substituted or unsubstituted pyrimidine or imidazole bases is included and/or a counter ion is included to maintain charge neutrality.

In one or more embodiments, the catalyst is a compound

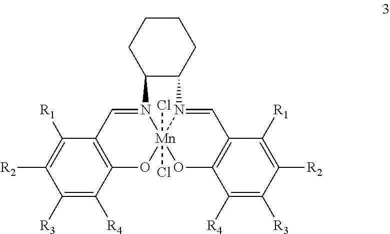

wherein R¹ is the same or different and is selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,

R² is the same or different and is selected from the group consisting of H, Cl, Br, CH₃, SO₃⁻, CN, [N(R′)₃]^{|}, COOR′, —OCONR′₂, —OMOM, CON—R′, CONR′₂, CH═NR′, SO₂NR′₂, SO₂R, CF and NO₂,

wherein R′ is H or a C1-C6 alkyl,

M is a transition metal, such as Fe, Zn, Co, Ni, Cu, Mn, Rh, Mg, Ru, Pt, and Pd,

and optionally wherein one or more axial ligands X selected from the group halogens (F, Cl, Br), OH, OCl, CO, [N(R′)₃]⁺, substituted or unsubstituted pyrimidine or imidazole bases is included and/or a counter ion is included to maintain charge neutrality.

In one or more embodiments, the catalyst is present at less than 5% wt/wt catalyst/organic substrate.

In one or more embodiments, the catalyst is present at less than 1% wt/wt catalyst/organic substrate.

In one or more embodiments, the catalyst is present at less than 0.5% wt/wt catalyst/organic substrate.

In one or more embodiments, the catalyst is a homogenous catalyst.

In one or more embodiments, the catalyst is a heterogeneous catalyst.

In another aspect, a method of making a metalloporphyrin includes combining a free porphyrin base and a metal source in a solvent to form a reaction mixture; and subjecting the reaction mixture to microwave energy to effect insertion of the metal from the metal source into the porphyrin.

In one or more embodiments, the metal source is selected from sources for iron, nickel, cobalt, ruthenium, manganese and rhodium.

### BRIEF DESCRIPTION OF THE DRAWINGS

The invention is described with reference to the following figures, which are presented for the purpose of illustration only and are not intended to be limiting.

FIG. 1 is a schematic illustration of a continuous flow process for the depolymerization of lignin according to one or more embodiments.

FIG. 2 is a sketch of a supported catalyst according to one or more embodiments of the invention.

FIG. 3 is a sketch of a supported catalyst according to one or more embodiments of the invention.

FIG. 4 is a schematic illustration of solid supports for use with a depolymerization catalyst according to one or more embodiments of the invention.

FIG. 5 is a thin layer chromatogram (TLC) 15 minutes after treatment of red dye (A), yellow dye (B) and black dye (C) with meso-tetrakis(2,6-dichlorophenyl)β-octabromo porphinato iron (III) chloride [Octachloro Octabromo Fe+3 TPP] illustrating the decomposition of the dye.

FIG. 6 is thin layer chromatogram (TLC) 1.3 hours after treatment of red dye (A), yellow dye (B) and black dye (C) with meso-tetrakis(2,6-dichlorophenyl)β-octabromo porphinato iron (III) chloride [Octachloro Octabromo Fe+3 TPP] illustrating the decomposition of the dye.

FIG. 7 is the uv-vis spectrum for the Jacobsen Mn Catalyst.

FIG. 8 is the uv-vis spectrum for the Modified Jacobsen Co catalyst.

FIGS. 9A-9B are the uv-vis spectrum for the blue dye (A) before and (B) after reaction with the Modified Jacobsen Mn catalyst in an oxidative decomposition reaction according to one or more embodiments.

FIGS. 10A-10B are the uv-vis spectrum for the black dye (A) before and (B) after reaction with the Modified Jacobsen Mn catalyst in an oxidative decomposition reaction according to one or more embodiments.

FIGS. 11A-11B are the uv-vis spectrum for the red dye (A) before and (B) after reaction with the Modified Jacobsen Mn catalyst in an oxidative decomposition reaction according to one or more embodiments.

### DETAILED DESCRIPTION

In one aspect, metalloporphyrins or salen complexes are used as efficient catalysts for the destructive oxidation of organic compounds.

The present invention is directed to the large scale catalytic oxidation of an organic substrate, in particular where the organic substrate is undesired or toxic. In other aspects, the invention is directed to the destructive oxidation of an organic substrate. As used herein, “destructive oxidation” is used to refer to a process that facilitates the decomposition of an organic compound by oxidation using catalytic process. As used herein “substrate” refers to the organic compound on which the catalyst acts. In certain embodiments, the decomposition products are benign or less toxic than the substrate organic compound. In other embodiments, the decomposition products are recovered as useful reaction products of the decomposition reaction. In preferred embodiments, the organic substrate is a lignin biopolymer. In other embodiments, the organic substrate includes molecules having unsaturated moieties such as alkenes C═C, alkynes, or azo derivatives. The unsaturated moieties can preferably be in conjugation with each other.

In one or more embodiments, a method is provided for the environmental remediation of organic compounds. Compositions containing toxic organic compounds can be degraded by oxidation to less toxic or benign decomposition products. The process can be carried out with or without prior processing of the composition containing the toxic organic compounds. Exemplary toxic organic compounds that can be degraded into more environmentally friendly components include organic dyes used in the textile industry.

In one or more embodiments, a method is provided for the degradation of polymers. Polymers are persistent in our environment and are stable to many chemical reactions. Polymers are subjected to oxidative degradation to break the polymers into smaller units. In some embodiments, the polymers are degraded into component monomers or oligomers. Exemplary polymers that can be degraded into lower molecular components include polymers having moieties within their backbone that are susceptible to oxidation. Lignin is a complex organic compound found in all plants and woody biomass that binds to cellulose fibers. In one particular embodiment, organic polymers such as lignins can be degraded into its component phenols and aromatic alcohols. The degradation of the lignin polymer also leads to the release of cellulose, hemicellulose and other components of the biomass.

In another aspect, destruction oxidation of biomass provides access to and chemical conversion of essential oils derived from biomass such as algae and eucalyptus. Essential oils, like all organic compounds, are made up of hydrocarbon molecules and can further be classified as terpenes, alcohols, esters, aldehydes, ketones and phenols. These can be converted to commodities and also to alkanes such as dodecane that are components of jet fuels. Monoterpenes are found in nearly all essential oils and have a structure of 10 carbon atoms and at least one double bond. The 10 carbon atoms are derived from two isoprene units. They react readily to air and heat sources.

The destructive oxidation can be carried out in organic or aqueous systems or mixtures thereof. The solvent in which the above reactions are carried out may be any solvent known to those skilled in the art which does not interact unfavorably with the synthetic metalloporphyrin and/or the co-oxidizing reagent. The organic substrate is usually treated in an aqueous system, however, small amounts of organic such as ethyl acetate, isopropyl acetate or other water miscible solvent such as tetrahydrofuran can be included to improve catalyst solubility. This is of particular use when a homogeneous catalyst is used. Exemplary solvents include water miscible solvents such as ethyl acetate, isopropyl acetate, tetrahydrofuran. Reaction can be carried out in systems containing 0-100% water, the balance including a water miscible solvent. Exemplary solutions include about 1:1 water:ethanol, 1:1 water:ethyl acetate and 1:2 water:methanol. In a heterogeneous system using supported catalysts, the system can be substantially or primarily aqueous. Non-polar solvents can also be used, such as toluene, dodecane and the like.

The amount to solvent used is not critical, as long as sufficient solvent is used to provide a paste or slurry or mixture or suspension of the organic substrate. In some embodiments, that organic substrate will have sufficient solubility to form an aqueous solution. However, use of supported catalysts eliminates the need for the organic substrate to be solubilized before processing. In homogeneous catalysis, the solvent should be of an amount and composition to solubilize the catalyst and distribute the catalyst in the biomass mixture. In heterogeneous systems, the mixture should be of a viscosity to allow the organic substrate to flow through and contact the supported catalyst.

The concentration of catalyst with such immense turnovers is miniscule. According to the present invention, the catalyst complex is added to the biomass at less than 5%, less than 1%, less than 0.5% wt/wt dry mass of organic substrate. Moreover, with immobilization or encapsulation, the catalyst levels can be even further reduced.

The oxidants that are used are effective in neutral to mildly alkaline conditions and the pH is maintained at this level throughout. Oxidants span a large variety from organic and inorganic peroxides, such as hydrogen or benzoyl peroxide, peracids such as 3-chloroperoxybenzoic acid (m-CPBA), hypochlorites such as sodium hypochlorite, exogenous oxygen donor molecules such as iodosyl benzenes (PhIO), inorganic salts such as potassium hydrogen persulfate, 2,6-dichloropyridine-N-oxide, tetra propyl perruthenate (TPP), ozone and molecular oxygen derived from moist air. The reaction is maintained at low/ambient temperatures, e.g., less than 100° C. or less than 70° C. or less than 60° C.

The reaction can be run as a batch process or continuously. All of the compounds can also be added continuously. Stirring of the reaction mixture may be employed. In certain embodiments, the process provides a treated solution that can be processed without additional chemical modification. In other embodiments, the oxidative degradation process produces a water soluble product that can be easily further processed.

In one or more embodiments, the catalyst is a supported catalyst and the oxidative decomposition process is run in a continuous flow process. Product separation is simplified when a supported catalyst is used and obviates tedious distillations or extractions.

Specific applications are described in the following examples, which are not intended to be limiting of the invention.

### Depolymerization of Persistent Organic Polymers

Oxidative destruction can also be used to breakdown many types of polymers. Exemplary polymers that serve as substrates for catalytic oxidative destruction include polyethylene, polypropylene, polystyrene, polyurethane and polyepoxy polymers. Such plastics are difficult to decompose and pose a serious problem in landfills, taking 100s or 1000s of years to decompose. In addition, billions of pounds of plastic can be found in swirling convergences making up about 40 percent of the world's ocean surfaces. The destructive oxidation process described herein can be used to breakdown plastics into smaller components that can be turned into useful products or that can be disposed of more readily.

Prior to depolymerization, the plastic can be prepared by chopping or grinding to reduce size. Undesirable components, such as metals, glass, dust, dirt can be removed before processing. In other embodiments, the plastics are ground to a powder.

The depolymerization of plastics is initiated by single one-electron oxidations of the substrate that are sustained catalytically by the catalyst. The catalyst will be reactive to moieties that are both saturated and unsaturated, such as the bonds found in urethanes or polystyrenes, or that can undergo further oxidation, such as in polyalkoxy polymers. The catalyst allows rapid and efficient degradation of plastics using very little catalyst. The use of the catalysts described herein provides high efficiency, rapid reaction rate, large turnover numbers that provide for the stoichiometric degradation of plastics in commercially meaningful amounts.

### Depolymerization of Lignin to Obtain Useful Chemical Decomposition Products

Lignin is found in cell walls in a mixture with cellulose and hemicellulose polymers. After cellulose, it is the most abundant carbon-based material on the earth. Yet, between 40 to 50 million tons are produced as waste each year. Processes that convert this non-usable waste into useful chemical products are desired.

Lignin is a cross-linked racemic macromolecule and a complex three dimensional polymer containing a variety of functional groups and structural features all derived from the polymerization of the highly oxygenated monomeric phenyl propenoid unit. It is relatively hydrophobic and aromatic in nature. Lignin is a copolymer of three different phenylpropane monomer units, namely para-coumaryl alcohol, coniferyl alcohol, and sinapyl alcohol. The degree of polymerization in nature is difficult to measure, since it is fragmented during extraction and the molecule consists of various types of substructures that appear to repeat in a haphazard manner. Different types of lignin have been identified depending on the means of isolation. A widely accepted schematic structure of lignin is that proposed by Adler, E. Wood Science and Technology 11(3):169-218. (1977), which is shown below. Although the proportion of these linkages varies according to the type of wood, typically more than two-thirds of the linkages in lignin are ether linkages. Hardwood lignin contains about 1.5 times more b-O-4-linkages.

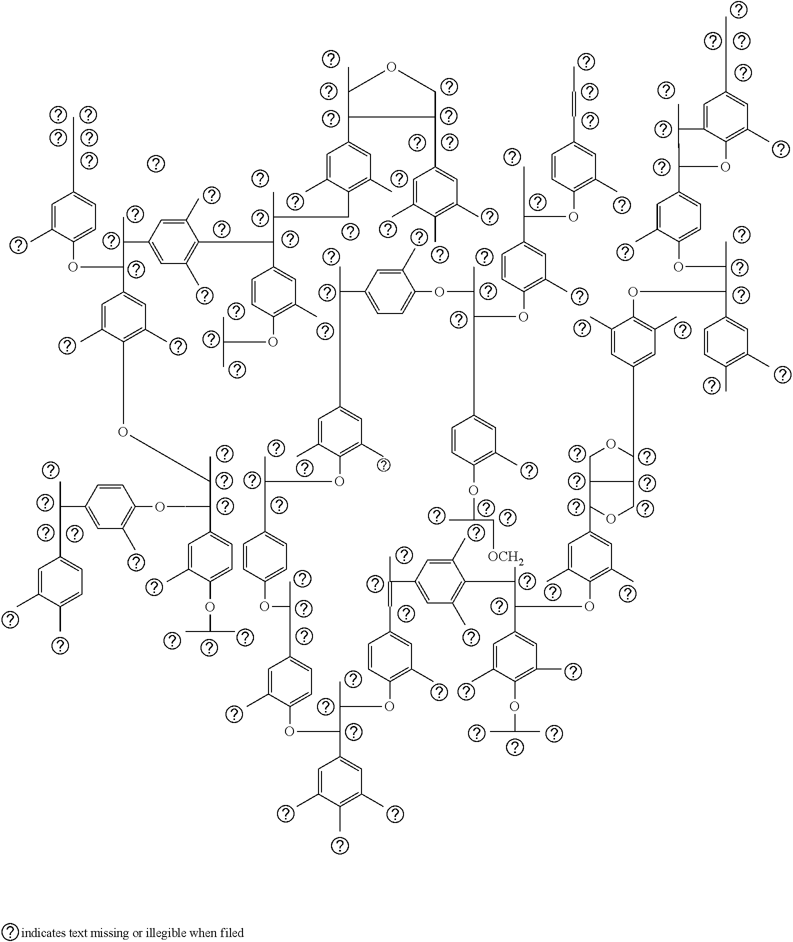

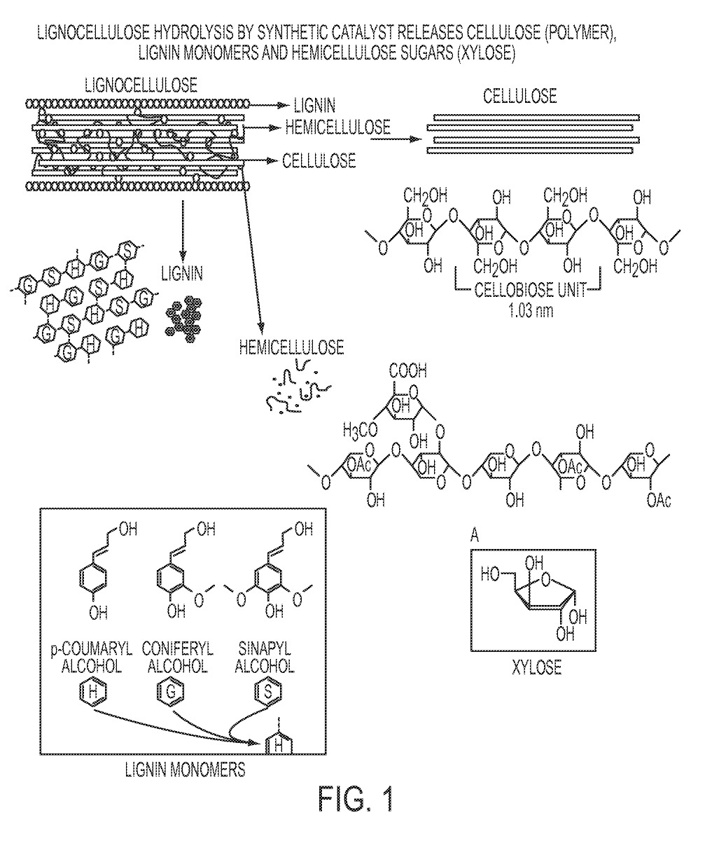

Depolymerization of lignin is accomplished by treatment with a catalyst for the decomposition of lignin into its component monomers and chemically modified derivatives thereof, such as phenols, para-coumaryl alcohol, coniferyl alcohol, and sinapyl alcohol. In addition polymers such as hemicellulose and cellulose are released from the complex organic substrate as the lignin is decomposed. A schematic illustration of the catalytic depolymerization of lignocellulose is shown in FIG. 1. As the lignin polymer is broken down, the lignin is reduced to its component monomers, phenols, para-coumaryl alcohol, coniferyl alcohol, and sinapyl alcohol. In addition, celluloses and bound up in the organic substrate are released.

In other embodiments, treatment of biomass by depolymerization of lignin also provides access to other useful components. Some sources of lignin also include high levels of terpenes. For example, in addition to lignin, Eucalyptus and lemon grass biomass contain a large number of terpenes and terpenoid products. This also provides terpenes and other raw materials that can be further processed to provide commodity chemicals such as 1-octanol and 1-octene. The chemical structures are much closer to octanol: this could offer a facile entry into octanol. The lignin contained in these plants is of course depolymerized as usual to release a mixture of celluloses and hemicelluloses: in addition the terpenes are liberated. These can be separated easily; their structures are much closer to octane and they can be converted to octane in a facile manner by organic reactions such as hydrogenation, and olefin metatheses.

The depolymerization of lignin is initiated by single one-electron oxidations of the substrate that are sustained catalytically by the catalyst. The catalyst allows rapid and efficient degradation of lignin using very little catalyst. The use of the catalysts described herein provides high efficiency, rapid reaction rate, large turnover catalysts that provide for the degradation of lignin in commercially meaningful amounts.

In one or more embodiments, depolymerization of lignin or lignin related compounds takes place using a catalyst selected from the group of metallotetraphenylporphyrins, metallophthalocyanines and metallosalen complexes and an oxidizing agent. In one or more embodiments, the catalyst is one or more of sterically hindered and electronically activated metalloporphyrins, phthalocyanines or salen complexes. In one or more embodiment, the catalyst is one or more of substituted metalloporphyrins, phthalocyanines or salen complexes that are substituted with electron withdrawing groups to electronically activate the metal center of the catalyst. In one or more embodiments, the electron withdrawing groups are located so as to provide steric hindrance and/or steric strain to the metal center of the catalyst. In one or more embodiments, the catalyst is one or more of compound 1, compound 2 or compound 3.

The lignin source can be a purified lignin or it can be a biomass source. Biomass is a material that is derived from living, or recently living biological organisms. Biomass often refers to plant material, however by-products and waste from livestock farming, food processing and preparation and domestic organic waste, can all form sources of biomass. Biomass includes bagasse, leafy or woody biomass, or any other biomass source. Exemplary biomass includes sugar cane, banana, cocoanut, eucalyptus or lemon grass, neem, wood (saw dust, wood chips, bark, etc.). Woody biomass is the accumulated mass, above and below ground, of the roots, wood, bark, and leaves of living and dead woody shrubs and trees and typically contains the greatest amount of lignin. Common sources of woody biomass typically come from harvesting and foresting operations. Prior to depolymerization, the biomass can be prepared by chopping or grinding the biomass to reduce size. Undesirable components, such as dust, dirt can be removed before processing. In other embodiments, the biomass is dried prior to grinding and the biomass provided as a dried powder.

Lignin can be extracted from biomass using known methods. Purified lignin or a biomass product enriched in lignin is obtained by extraction of soluble components from the biomass source. See, for example, the procedures described by Romualdo S. Fukushima and Ronald D. Hatfield, Journal of agricultural and food chemistry, July 2001, Vol. 49, Number 7, pp. 3133-3139. In one exemplary extraction process, biomass is subjected to sequential extraction with water, ethanol, chloroform, acetone, and acidic dioxane. The resulting residue is dried to provide a source of lignin that can be depolymerized according to one or more embodiments described herein.

The depolymerization can be carried out in organic or aqueous systems or mixtures thereof. The solvent in which the above reactions are carried out may be any solvent known to those skilled in the art which does not interact unfavorably with the synthetic metalloporphyrin and/or the co-oxidizing reagent. The biomass is usually treated in an aqueous system, however, small amounts of organic such as ethyl acetate or other water miscible solvent can be included to improve catalyst solubility. This is of particular use when a homogeneous catalyst is used. Exemplary solvents include water miscible solvents such as ethyl acetate, isopropyl acetate, tetrahydrofuran. Reaction can be carried out in systems containing 0-100% water, the balance including a water miscible solvent. Exemplary solutions include about 1:1 water:ethanol, 1:1 water:ethyl acetate and 1:2 water:methanol. In a heterogeneous system using supported catalysts, the system can be substantially or primarily aqueous. Non-polar solvents can also be used, such as toluene, dodecane and the like. Water immiscible solvents can be used with phase transfer catalysts that facilitate reaction; this is exemplified by use of PTCs such as tetra n-butyl ammonium bromide.

The amount of solvent used is not critical, as long as sufficient solvent is used to provide a paste or mixture or suspension of the biomass. In homogeneous catalysis, the solvent should be of an amount and composition to solubilize the catalyst and distribute the catalyst in the biomass mixture. In heterogeneous systems, the mixture should be of a viscosity to allow the biomass to flow through and contact the supported catalyst.

The concentration of catalyst with such immense turnovers is miniscule. According to the present invention, the catalyst complex is added to the biomass at less than 5%, less than 1%, less than 0.5% wt/wt dry mass of biomass. Moreover, with immobilization or encapsulation, the catalyst levels can be even further reduced.

The oxidants that are used are effective in neutral to mildly alkaline conditions and the pH is maintained at this level throughout. Oxidants span a large variety from organic and inorganic peroxides, such as hydrogen or benzoyl peroxide, peracids such as 3-chloroperoxybenzoic acid (m-CPBA), hypochlorites such as sodium hypochlorite, exogenous oxygen donor molecules such as iodosyl benzenes (PhIO), inorganic salts such as potassium hydrogen persulfate, 2,6-dichloropyridine-N-oxide, ozone and molecular oxygen derived from moist air. The reaction is maintained at low/ambient temperatures, e.g., less than 100° C. or less than 70° C. or less than 60° C.

The reaction can be run as a batch process or continuously. All of the compounds can also be added continuously. Stirring of the reaction mixture may be employed. In certain embodiments, the process provides a treated solution that can be processed without additional chemical modification. For example, the treated solution can be further treated to isolate the phenols and also the hemicellulose and cellulose; the latter can be converted to into sugars such as glucose or xylose.

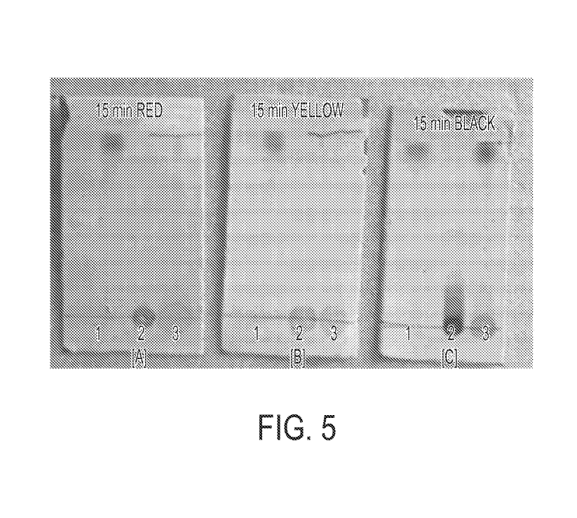

In one or more embodiments, the catalyst is a supported catalyst and the depolymerization process is run in a continuous flow process. An exemplary continuous flow process is shown in FIG. 5. Product separation is simplified when a supported catalyst is used and obviates tedious distillations or extractions.

Once decomposed, the desired small organic molecules can be separated from the decomposition mixture using conventional processes. For example, phenols and aromatic alcohols can be obtained by extraction of the reaction mixture with sodium hydroxide or aqueous alkali.

The reaction products from the destructive degradation of lignin-containing biomass has many uses. Extensions to production of aviation and jet fuels can readily be envisioned. Alkanols such as octanols can be dehydrated to alkenes such as 1-octene that can converted by olefin metathesis to commodity chemicals.

Biomass comprises three major components: lignin, cellulose and hemicelluloses. Lignin is a copolymer of three different phenylpropane monomer units (monolignols), methoxylated to various degrees to produce para-coumaryl alcohol, coniferyl alcohol, and sinapyl alcohol. These can be used as perfumery chemicals, anti-oxidants and myriad other applications such as synthesis of vanillaldehyde, terephthalic acid by chemocatalytic processes

Lignocellulosic materials contain large amount of polysaccharides derived from C6 and C5 sugars. 60-70% of these sugars are entangled within lignin and cannot be used. With our technology, cellulose and hemicelluloses are liberated from lignin using mild reaction conditions. The oligosaccharides can be converted to the sugars amenable for production of bio-alkanols and also to a variety of high value chemicals such as levulinic acid, gama-valerolactone, and furfural derivatives.

### Detoxifying Organic Dyes

Toxic chemicals from dyes create severe environmental havoc. Effluents releasing from dyeing industries directly affect the soil, water, plant and human life. Large amounts of water are used to flush conventional synthetic dyes from garments and then this waste water must be treated to remove the heavy metals and other toxic chemicals before it can be returned to water systems, sewers and rivers. The environmental impact of the textile industry is significant: It uses 9 trillion liters of water and 1 trillion KW hours of electricity. The industry is responsible for 10% of the world's carbon footprint.

Currently bleaching of the vat and other dyes is carried out at 95 degrees C. and a pH of 12 or higher. Large scale catalytic oxidation using the porphyrin or salen metallocatalysts described herein circumvents this hazardous process by oxidizing the dyes, breaking the extended chromophore. It is expected that energy consumption can be reduced by half due to lower treatment and rinsing temperatures. No neutralization is required and water usage can be reduced.

Organic dyes are colored, ionizing, aromatic organic compounds. Organic dyes include atomic configurations that contain delocalized electrons. Usually they are represented as nitrogen, carbon, oxygen and sulfur that have alternate single and double bonds. For example, organic dyes include aromatic groups and other delocalized resonance structures, such as quinonoid rings, —C═C—, —C═N—, —C═O— and —N═N—. Such moieties or chemical groups can be oxidized to break down or degrade the dyes. The proposed chemistry that is likely occurring is primarily oxidation, hydroxylation and epoxidation.

The porphyrin mediated oxidation of dye effluent can help reduce the toxicity of the dyes. Detoxifying organic dyes can be conducted according to the following reaction:

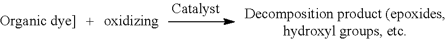

The reaction is very rapid indicating that complete oxidation of the dye has taken place. The starting organic dyes have low solubility in water, requiring large amounts of water for dilution and removal. The resulting degradation products are water soluble, which allows them to be processed using less water. In addition, the catalyst is highly effective, so that large amounts of dye can be effectively degraded using very little catalyst.

### Catalysts

Synthetic metalloporphyrins (SMP) have received a lot of recent attention as mimics of numerous enzymes and models of oxidative catalysts in biological systems. In addition to serving as models for peroxidases and particularly the ligninases, metalloporphyrins have also found utility as model systems for studies of the oxidative metabolism of drugs. Highly halogenated metalloporphyrin complexes have been show to function as mimics of the ligninases. The ligninases are heme proteins which are produced by fungi and used in nature to assist in the degradation of lignin. However, unsubstituted metalloporphyrins are usually poor catalysts because they are degraded by the oxidizing environments in which they operate.

The metalloporphyrin and salen complexes described herein demonstrate efficient, rapid and catalytic degradation of organic compounds. In specific examples of this process, the metalloporphyrin and salen complexes described herein demonstrate efficient, rapid and catalytic depolymerization of lignin and degradation of organic dyes. In some embodiments, the catalyst is used to degrade organic dyes to reduce toxicity and improve water solubility. In other embodiments, the catalyst is used to catalytically decompose lignin into its substituent monomers and oligomers. High turnover numbers and long catalyst life are observed. In one or more embodiments, turnover numbers of more than 10,000, more than 50,000 or more than 100,000 can be obtained. The catalyst is one or more substituted meso-tetraphenylporphyrins, phthalocyanines or salen complexes that are sterically protected and electronically activated. The structural scaffolds incorporate the tetra aza macrocycle (metalloporphyrin) or the salen complexes into the primary structure. Further structural variants including modulation of the macrocycle (number of rings), the substitution pattern at the periphery of the aromatic rings ranging from di to penta substitution, the substitution on the β-pyrrole hydrogens, the complexing metal ions, the choice of axial ligands, the inorganic counter-ions and the polymer used for immobilization at specified binding sites, can be introduced to modulate the catalytic properties of the catalyst.

Steric bulk is introduced into the scaffold by substitution at the aromatic and pyrrole ring sites in tetraphenylporphinato complexes and on the aromatic ring sites in phthalocyanines and salen complexes. In preferred embodiments, the ring substituents provide both steric bulk and electronic activation. Thus, substituents that provide both steric strain and electron withdrawing properties to the catalyst are associated with improved catalyst lifetime and activity. In one or more embodiments, the ortho-position of the phenyl groups in the tetraphenylporphinato complexes are substituted with electron withdrawing groups “E”. In one or more embodiments, both the ortho-position of the phenyl groups and the β-position of the pyrrole groups in the tetraphenylporphinato complexes are substituted with electron withdrawing groups “E”. In one or more embodiments, the alpha-positions on the aromatic groups of the phthalocyanines and salen complexes are substituted with electron-withdrawing groups. Suitable electron withdrawing groups are strong electron withdrawing, coordinating or chelating groups that have the effect of increasing the kinetic acidity of protons in the adjacent positions, reducing electron availability at carbon atoms. Such high electronegativity is transmitted to the central metal atom in a porphyrin, salen or other aza macrocycle. Exemplary electron withdrawing groups include —OCONR′₂, —OMOM (Methoxymethyl ether), —CON—R′, —CONR′₂, —CH═NR′, —SO₂NR′₂, —SO₂tBu, —CN and —CF, where R′ is H or a C1-C6 alkyl and M is a metal.

In addition, it is preferred that the substituents at the ortho-position of the phenyl groups and/or the β-position of the pyrrole groups in the tetraphenylporphinato complexes have a steric bulk that introduces steric bulk into the molecule. In one or more embodiments, the substituents at the ortho-position of the phenyl groups and/or the β-position of the pyrrole groups in the tetraphenylporphinato complexes have a steric bulk that is at least as large as a chloride anion. In one or more embodiments, the substituents at the alpha-positions on the aromatic groups of the phthalocyanines and salen complexes have a steric bulk that is at least as large as a chloride anion.

Exemplary metalloporphyrin catalysts for use in the depolymerization of lignin include meso-tetraphenyl porphinato complexes as shown by compound 1

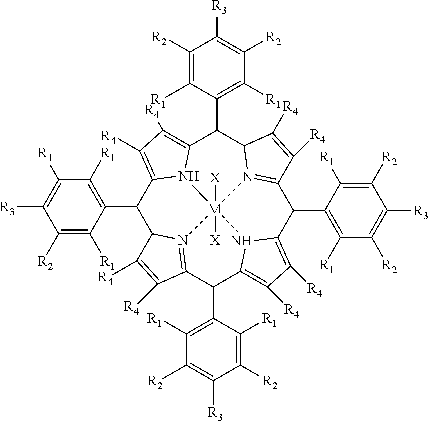

in which any of the R¹ are the same or different and are selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂, any of the R², R³ or R⁴ are the same or different and are selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂, wherein R′ is H or a C1-C6 alkyl and in which M is a transition metal, such as Fe, Zn, Co, Ni, Cu, Mn, Rh, Mg, Ru, Pt, and Pd. In addition, the compound can include axial ligands X (ligand complexed to the metal center above or below the porphyrin plane. Exemplary axial ligands X include halogens (F, Cl, Br), OH, OCl, CO, [N(R′)₃]⁺, substituted or unsubstituted pyrimidine or imidazole bases. In some instances, a counter ion is included to maintain charge neutrality.

Sterically unprotected metalloporphyrins are oxidatively labile, and are not stable to multiple turnovers. Introduction of halogens onto the aryl groups (of meso-tetraphenylporphyrins) and on the β-pyrrolic positions of the porphyrins increases the turnover of catalytic reactions by decreasing the rate of porphyrin destruction. In addition, the combined electronegativities of the halogen substituents are transmitted to the metal atom making the corresponding oxo-complexes (formed during catalytic oxidation) more electron deficient and thus more effective oxidation catalysts. Sulfonation with H₂SO₄/SO₃ places a sulfonic acid group in each of the phenyl rings thereby adding further electron withdrawing groups to impart stability and water solubility: it also provides a point of attachment for immobilization.

In addition to providing dramatic electronic activation the beta-halogens also cause a significant change in the conformation of the porphyrin ring. Bulky groups on the beta-positions of meso-tetraphenylporphyrins can cause the normally flat aromatic porphyrin to take up a saddle shape where the four pyrrole rings alternately point up and down with respect to the mean porphyrin plane. The perhalogenated porphyrins take up similar conformations. This saddle conformation results in even greater steric protection than the planar conformation. Thus “perhalogenation” provides both steric protection and electronic activation of metallo tetraphenylporphyrins making them excellent biomimetic catalysts.

In one or more embodiments, the sterically protected electronically activated metalloporphyrins include meso-tetraphenylporphyrins having electron withdrawing substituents at the ortho-aryl and optionally at the β-pyrrole positions. Exemplary electron withdrawing substituents include Cl, Br, CN, SO₃H and NO₂. Exemplary metalloporphyrins include meso-tetrakis(2,6-dichlorophenyl)porphinato iron (III) chloride [Octachloro Fe+3 TPP] 1a, meso-tetrakis(2,6-dichlorophenyl)β-octachloro porphinato iron (III) chloride [Octachloro Octachloro Fe+3 TPP] 1b, and meso-tetrakis(2,6-dichlorophenyl)β-octabromo porphinato iron (III) chloride [Octachloro Octabromo Fe+3 TPP] 1c. and meso-tetrakis(2,6-dichloro, 3-sulfonatophenyl)β-octachloro porphinato iron (III) chloride [Octachloro Octachloro Tetrasulfonate Fe+3 TPP] 1d,

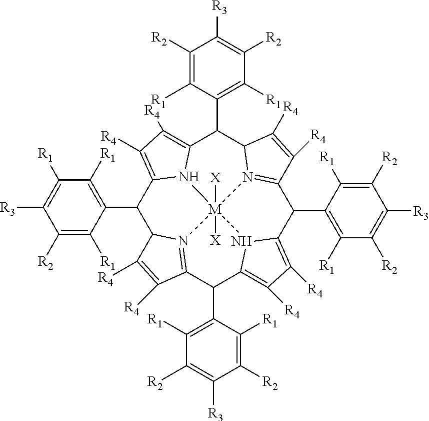

where compound 1a includes M=Fe, R1=Cl, R2=R3=H, R4=Cl; compound 1b includes M=Fe, X═Cl, R1=R4=Cl, R2=R3=H; compound 1c includes M=Fe, R1=Cl, R2=R3=H, R4=Br; and compound 1d (M=Fe, X═Cl, R1=Cl, one R2=H, and one R2=SO₃Na, R3=H, R4=Br.

Exemplary phthalocyanines (or tetrabenzotetraazoporphyrins) for use in the depolymerization of lignin include compound 2,

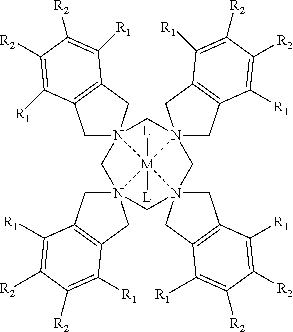

in which any of the R¹ are the same or different and are selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂, any of the R² are the same or different and are selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂, wherein R′ is H or a C1-C6 alkyl and in which M is a transition metal, such as Fe, Zn, Co, Ni, Cu, Mn, Rh, Mg, Ru, Pt, and Pd. In addition, the compound can include axial ligands X (ligand complexed to the metal center above or below the porphyrin plane. Exemplary axial ligands X include halogens (F, Cl, Br), OH, OCl, CO, [N(R′)₃]⁺, substituted or unsubstituted pyrimidine or imidazole bases. In some instances, a counter ion is included to maintain charge neutrality.

As in the case for meso-tetraphenylporphyrins, introduction of halogens onto the benzo groups (of the phthalocyanines) activates the oxidation reaction by increasing the deficiency of the coordinated metal. The combined electronegativities of the halogen substituents are transmitted to the metal atom making the corresponding oxo-complexes more electron deficient and thus more effective oxidation catalysts. Sulfonation with H₂SO₄/SO₃ places a sulfonic acid group in each of the benzo rings thereby adding further electron withdrawing groups to impart stability and water solubility: it also provides a point of attachment for immobilization. In addition, the bulk of the benzo substituents also provide steric strain protection to the porphyrin ring. Thus “perhalogenation” provides both steric strain and electronic activation of phthalocyanines making them excellent biomimetic catalysts.

In one or more embodiments, the sterically protected electronically activated metalloporphyrins include phthalocyanines having electron withdrawing substituents at the ortho-aryl positions. Exemplary electron withdrawing substituents include Cl, Br, CN, SO₃ and NO₂. Exemplary phthalocyanines include compound 2a and 2b,

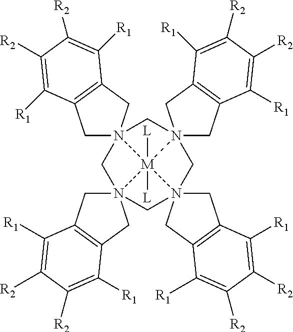

where compound 2a has R₁═R₂═Cl, and compound 2b had R₁═R₂═H.

Salen ligands are Schiff bases, usually prepared by the condensation of a salicylaldehyde with an amine. Exemplary salen complex catalysts for use in the depolymerization of lignin include compound 3,

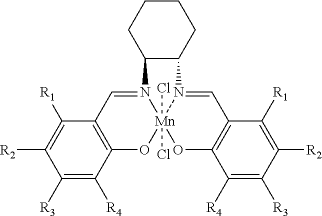

in which any of the R¹ are the same or different and are selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂, any of the R² are the same or different and are selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂, wherein R′ is H or a C1-C6 alkyl and in which M is a transition metal, such as Fe, Zn, Co, Ni, Cu, Mn, Rh, Mg, Ru, Pt, and Pd. In addition, the compound can include axial ligands X (ligand complexed to the metal center above or below the porphyrin plane. Exemplary axial ligands X include halogens (F, Cl, Br), OH, OCl, CO, [N(R′)₃]⁺, substituted or unsubstituted pyrimidine or imidazole bases. In some instances, a counter ion is included to maintain charge neutrality.

The chelating salen ligand is tetradentate, meaning it binds to the central manganese metal through four bonds, one to each oxygen and nitrogen atom of the salen backbone. The catalytic properties of bulky water-soluble Co-, Cu-, Fe- and Mn-salen complexes in the oxidation of phenolic lignin model compounds have been studied in aqueous water-dioxane solutions (pH 3-10). Mn catalysts were found to oxidize coniferyl alcohol in a same reaction time as horseradish peroxidase (HRP) enzyme and Mn and Co catalysts showed different regioselectivity suggesting a different substrate to catalyst interaction in the oxidative coupling. When the oxidation of material more relevant to plant polyphenolics was studied, the results indicated that the complexes catalyze one- and two-electron oxidations depending on the bulk of the substrate: this is of course, the same mechanism as used by the metalloporphyrins and by the heme proteins such as hemoglobin and myoglobin.

In one or more embodiments, the sterically protected electronically activated salen complexes have electron withdrawing substituents at the ortho-aryl positions. Exemplary electron withdrawing substituents include Cl, Br, CN, SO₃ and NO₂. Exemplary salen complexes include compound 3a and 3b

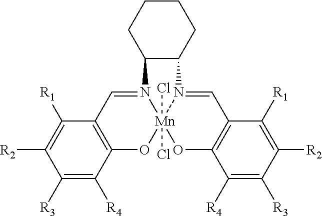

wherein compound 3a has M=Mn, X═Cl, R₂═Br, R₁═R₃═R₄′ and compound 3b has M=Mn, X═Cl, R₂═NO₂, R₁═R₃═R₄.

### Supported Catalysts

In each of the above catalyst complexes, the catalyst can be used as a homogeneous catalyst in a reaction mixture that includes the substrate for oxidative degradation. In other embodiments, the catalyst can be a heterogeneous catalyst. Heterogeneous catalysts are typically supported, which means that the catalyst is dispersed on, encapsulated in or attached to a second material that enhances the catalytic effectiveness of the catalyst or minimizes its cost. A major attraction of supported catalysts is that the supported species can be separated easily, for example by filtration, from the unreacted starting materials and reaction products. This easy separation can greatly simplify product isolation procedures, and it may even allow the supported reactions to be automated. Because it is possible to reuse or recycle supported reactants and because they are insoluble and nonvolatile, they are easily handled, and easily recovered. In addition, supported reactants are also attractive from an environmental point of view. Lastly, the catalyst support permits adaptation of the process to a continuous flow process. Flow chemistry can be adapted to micro reactors that reduce waste and provide a ‘greener’ reaction process.

Suitable supports are porous materials with a high surface area. The solid support can include, for example, polymers, metal oxides and other ceramics such as silica, titania, calcium carbonate, zeolites, molecular sieves, clay and alumina, and carbons such as activated carbon or carbon nanotubes, polymers and sulfonated or fluorinated resins. In particular, the catalyst can be immobilized onto a polymer support using known techniques. Examplary polymer beads include polystryene, styrene-divinylbenzene, fluorinated polymers such as TEFLON, polyethylene glycol. In particular, polystyrene having an ave. molecular weight of 30 Kdaltons to 240 Kdaltons can be used. The polymers can be halogenated.

In order to tether the catalyst to the support, the catalyst is attached to a polymer support by reaction at one or more locations on the porphyrin or salen ring structure. The catalyst can be tethered directly or through a linker (such as an alkyl or polyalkoxy chains to the catalysts. In some embodiments, the catalytically active species are immobilized or encapsulated through chemical bonds or weaker interactions such as hydrogen bonds or donor-acceptor interactions. The aryl rings of the meso-tetrakis phenyl porphinato complexes and the benzo rings of the phthalocyanine complexes provide useful locations for functional groups that can link the catalyst to a solid support. Exemplary functional groups include amino, hydroxyl and sulfonate, sulfonyl, sulfonamide, carboxylate groups. Amino can be introduced onto the porphyrin ring or salen ring by direct nitration, followed by reduction to the corresponding amine. The amine is used as a functional group to link to reactive species a functionalized polymer support using well-known techniques. Scheme 1 shows a reaction pathway for the nitration and sulfonation reactions useful to generate reaction nitrate and sulfonate groups.

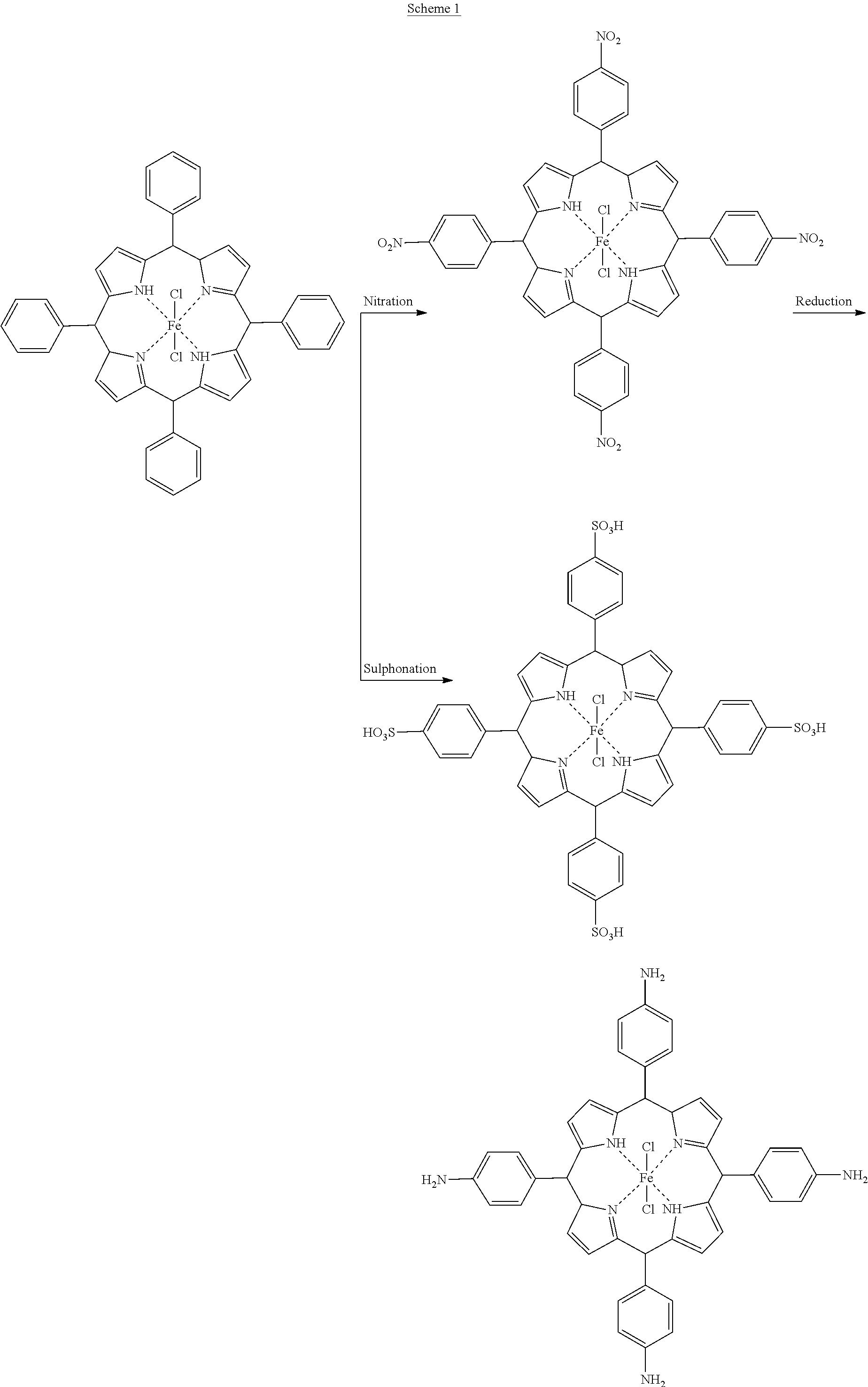

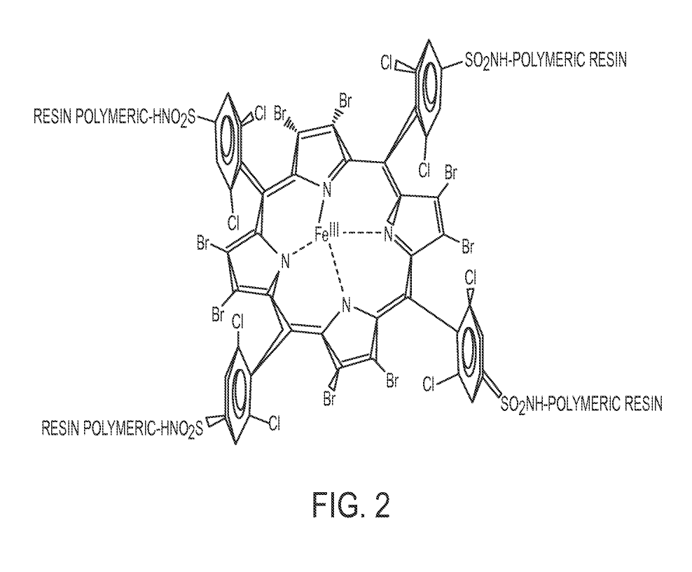

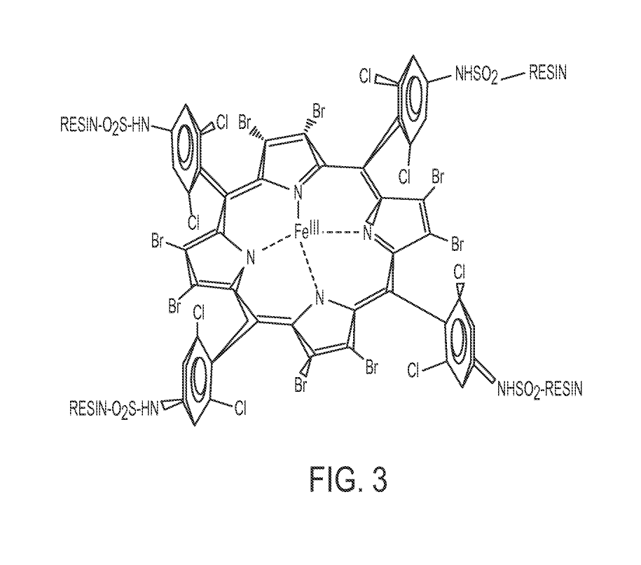

FIG. 2 shows a supported polymer complex using [Octachloro Octabromo Fe+3 TPP] 1c as the catalyst, attached to a polymeric resin solid support through sulfonamide groups (˜SO₂NH˜). Similarly, FIG. 3 shows a supported polymer complex using [Octachloro Octabromo Fe+3 TPP] 1c as the catalyst, attached to a polymeric resin solid support through aminosulfonato groups (˜NHSO₂˜).

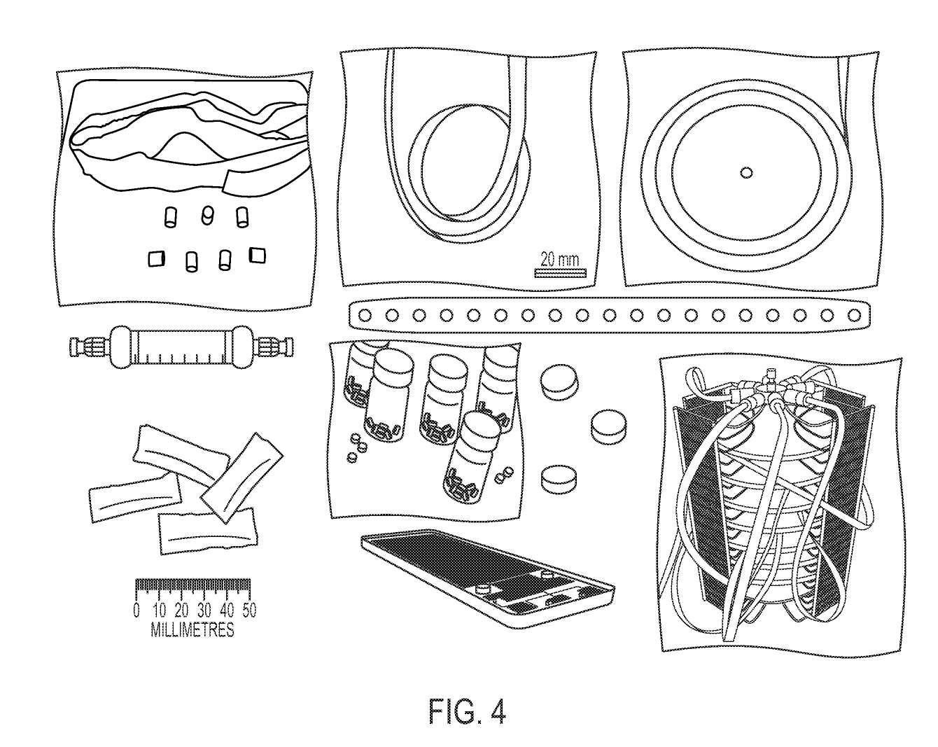

In other embodiments, the polymer support can be in the shape of a ribbon, gels, beads, strips, coils and the like. Polymer-supported catalyst can be prepared in the form of beads of about 50-100 micrometers diameter. The beads are functionalized (on the interior and/or exterior surfaces) with groups that react with and tether the catalyst to the solid support. The various form factors of the supported catalyst is shown in FIG. 4. The catalysts can be made into spheres, ribbons, flat sheets, immobilized matrices of one layer thickness, tubular coils, which are available in a range of materials. These are readily replaced or interchanged. Sections of coil are interchangeable with cartridges that can be loaded with solid supported catalysts or reagents.

In one or more embodiments, the catalyst can be supported using encapsulation technology. During catalytic oxidation using metalloporphyrins or metallophthalocyanines under homogeneous conditions, the system can encounter challenges such as catalysts separation, dimerization and catalyst destruction. These challenges can be avoided by using a polymer microencapsulated catalyst. Microencapsulation is a method for immobilizing catalysts onto polymers such as ionic resins and polystyrene on the basis of physical entrapment in the polymer matrix. The catalysts are firmly anchored through the electronic interactions between the π electrons of the benzene rings of the polystyrene-based polymers and the vacant d orbitals of the catalyst. This is an efficient and easy method for immobilization of commercially available metalloporphyrins and metallophthalocyanines onto polystyrenes in general, which gives stable, reusable and highly efficient catalysts for aerobic oxidation of alcohols and exhibit enhanced activity over their unencapsulated counterparts. The combination of polymer supported catalysts and molecular oxygen as sole oxidant constitutes an excellent process for the depolymerization of lignin.

The method of encapsulation was standardized using different types of polystyrene polymers as well as different metallophthalocyanines Metallophthalocyanines of iron, cobalt and copper have been successfully encapsulated on polystyrene matrices, rendering them highly dispersible in common organic solvents. An exemplary reaction scheme for the encapsulation of metallophthalocyanines is shown in Scheme 2.

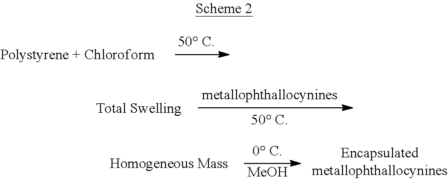

These immobilized systems have obvious advantages because the catalysts are more easily separated from products and recycled which is especially important when dealing with fairly expensive metalloporphyrins. Using this micro encapsulation technique (Scheme 2), metalloporphyrins of manganese have been successfully encapsulated in polystyrene matrix. The manganese porphyrins have been anchored onto polymer on the basis of physical envelopment by the polystyrene fibers, rendering them highly dispersible in common organic solvents.

### Synthesis of the Sterically Protected and Electronically Activated Metalloporphyrins

Methods for producing substituted metalloporphyrins are well known and can be readily employed in the preparation of the sterically hindered, electronically activated catalysts used in the catalytic oxidation of organic substrates. The synthetic metalloporphyrins may be prepared by known methods, wherein a suitable zinc-containing metalloporphyrin, such as meso-tetrakis(2,6-dihalophenyl)porphyrinato-zinc (II), wherein “halo” is chloro, bromo, fluoro, or iodo, is reacted with one of several active halogenating agents, followed by removal and replacement of the zinc by the desired active metal ion. They may also be prepared by an improved method for the preparation of a porphyrin-ring halogenated synthetic metalloporphyrin. wherein the halogenating agent may be a free halogen, such as Cl₂ or Br₂ in a suitable polar solvent such as methanol. ethanol. or the like.

Practical and efficacious methods of synthesizing porphyrins with halogens at the ortho-aryl and also the β-pyrrole positions are described in Scheme 3. The methodologies provide facile access to a large number of porphyrins in optimum yields and purity.

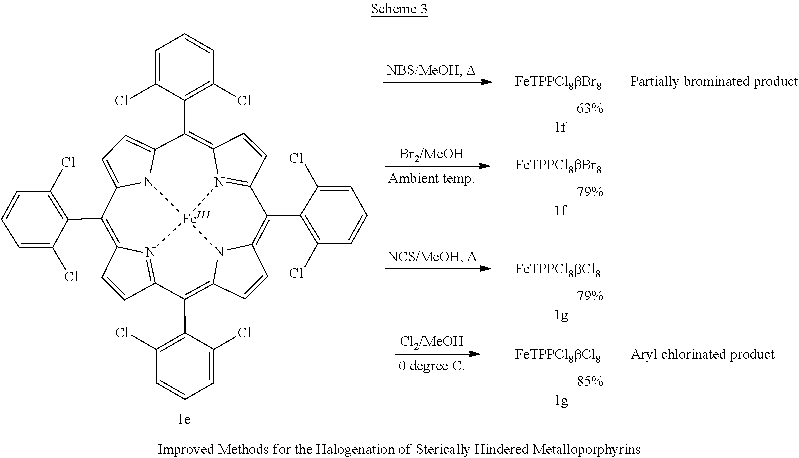

In other schemes, the Zn-metallized compound can be prepared using conventional methods such as halogenation of the tetrapyrrole base using dichlorobenzaldehyde and zinc acetate as the source of the complexing metal. See, Example 1 and Traylor, P. S.; Dolphin. D.; Traylor. T. G. J. Chem. Soc., Chem. Commun. 1984, 279 and M. S. Chorghade*, D. H. Dolphin*, D. Dupre, D. R. Hill, E. C. Lee and T. P. Wijesekara, “Improved Protocols for the Synthesis and Halogenation of Sterically Hindered Metalloporphyrins”, Synthesis, 1996, 1320. Further halogenation of the porphyrin ring structure is achieved by reaction with N-halo succinimides or elemental halogens. Once the appropriate halogenated porphyrin is obtained, a metalloporphyrin of the desired metal is prepared by demetallation with trifluoroacetic acid followed by insertion of the desired metal using known techniques by reaction with the appropriate metal salt.

It has been surprisingly shown that the electronically activated and sterically hindered metalloporphyrins disclosed herein can be synthesized rapidly and efficiently by metal insertion into a free porphyrin ring by heating the free base in the presence of a metal source under conventional microwave at low temperature, e.g., 750 W microwave at microwave length of 23450 MHz. The reaction occurs according to the general reaction:

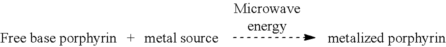

Exemplary metal sources include metal chlorides, such as iron chloride, copper chloride, and nickel chloride. Reactions are carried out in solution or in suspension and include subjecting the reaction mixture to microwave energy for a period of time. A range of microwave energy may be used.

Previously, free base porphyrins have been metallated with microwave irradiation. However, the porphyrins were neither sterically protected nor electronically activated. Previously metalized derivatives of the porphyrins had a flat and planar ring, where insertion is a facile process. The sterically hindered, electronically activated porphyrins used herein as catalysts, have a saddle-shaped puckered ring system; this puckering eliminates the planar central cavity and makes the deformed ring more difficult to metallate. Moreover, the electronic activation conferred on the ring has one additional problem related to metal insertion. Previously, insertion of metals such as Ru, Rh etc., in extended reflux in a very high boiling solvent such as decane xylene was unsuccessful. This process resulted in poor yield, as the Ru salt, serving as a Lewis acid and electrophile, methodically abstracted the halogens one after the other, degrading the compound to the tetraphenyl porphyrin. Using microwave energy insertion of Ru into a highly puckered core proceeds rapidly and with high yields.

Known methods can be used to prepare phthalocyanine and salen complexes.

The following examples are provided for the purpose of illustration and are not intended to be limiting of the invention, the full scope of which is set out in the claims that follow.

### Catalyst Synthesis

### EXAMPLE 1

### Synthesis Of Octachloro Iron (III) Chloride (meso-Tetrakis(2,6-dichlorophenyl)porphinato-iron(III) chloride) (1a)

1) Octachloro Zn-Complex (1h)

2,6-Dichlorobenzaldehyde (52.5 g; 0.30 mol), anhydrous Zn (OAc)2 (20 g; 0.11 mot), and 2,6-dimethylpyridine (150 mL) were placed in a 1 L round bottom flask fitted with a Soxhlet extractor surrounded by a reflux condenser. Anhyd. Na₂SO₄was placed in a 6×17 cm thimble as a drying agent and the mixture was heated. When the temperature reached 100° C., pyrrole (21.0 mL; 0.30 mol) was added drop wise (within 10 min); the condenser was removed, allowing the evaporation of some water, formed as droplets in the reaction. The condenser was replaced about 5 min later and the solution was refluxed for 6 h. After evaporating the solvent in vacuo the resulting tarry residue was triturated with toluene (375 mL) then CH₃OH (75 mL) was added; the mixture was allowed to stand in the refrigerator overnight. The porphyrin precipitated as its Zn complex mixed with zinc acetate. It was filtered, rinsed with a small amount of CH₃OH and dried under vacuum in a desiccator, yielding 12.83 g. The powder was purified by dispersing in hot water (500 mL) and stirring overnight, followed by filtering, washing with methanol and finally rinsing with pentane, affording the title compound 1h. The structure was confirmed by uv-vis, IR and ¹H NMR spectroscopy.

2) Demetallation of Octachloro Zn-Complex

The impure zinc complex (1.19 g; 1.25 mmol) was dissolved in CHCl3 (300 mL), TFA (10 mL) was added and the mixture was stirred; the progress of the reaction was monitored by UV-V1 S spectroscopy in CH2Cl2 after neutralization of the sample by Et3N. After 1 h the crude product was washed with H2O (350 mL), aq NaHCO3 (350 mL), H20 (2×250 mL), and dried (MgSO4). After filtration, the volume was reduced to about 100 mL and the compound was crystallized by addition of MeOH (50 mL) followed by partial evaporation of CHCl3, affording the title compound. The structure was confirmed by uv-vis, IR and 1H NMR spectroscopy.

### Oxidation of Chlorin Impurity

The chlorin-contaminated free base 1 (1.83 g; 2.06 mmol) was dissolved in pentene-stabilized CHCl₃ (600 mL) and heated to reflux in a 1-L round bottom flask. A solution of DDQ (1.6 g) in benzene (75 mL) was added drop wise over 20 min and the reflux was continued for 2 h, then stirred overnight at r. t. It was passed quickly through alumina (160 g) placed in a 7 cm wide sintered funnel. It was thoroughly rinsed with hot CHCl₃. The solution concentrated to 250 mL, and the compound was crystallized by addition of CH₃OH (200 mL) followed by slow evaporation of CHCl₃ affording oxidized product.

3) Metal Insertion [Synthesis of meso-Tetrakis(2,6-dichlorophenyl)porphinato-iron(llI) chloride].

The free base (1.22 g; 1.37 mmol), FeCl₂-4H₂O (2.73 g; 13.7 mmol), and DMF (400 mL) were degassed and refluxed under argon in a 1 L round bottom flask. The progress of the reaction was monitored by UV-Vis in CH₂Cl₂. Metal insertion was completed in 3 h. Heating was discontinued and the solution was stirred open to the air and allowed to cool at room temperature. After removing solid FeCl₃ by filtration, the solution was concentrated in vacuo to approximately 250 mL, and 5 N HCl (750 mL) was added; the hemin precipitated out as a brown solid from a clear yellow solution. After filtration, it was thoroughly washed with water and dried in a vacuum desiccator. This product was redissolved in CHCl₃ (150 mL) (the complex is significantly more soluble in chloroform than in dichloromethane) and purified by chromatography on silica (130 g; 5×17 cm). A trace of unreacted free base was eluted using CH₂Cl₂; it was followed by a trace of bluish-purple ring-opened by-product which was eluted using 2% CH₃OH in CH₂Cl₂ (v/v). The hemin was then eluted by increasing the concentration of CH₃OH to 5%. After evaporation to dryness, the complex was redissolved in CHCl₃ (150 mL), treated once with an equal volume of 5 N HCl, washed with water until neutral, and dried over MgSO₄. The hemin was recrystallized from CHCl₃/hexane. The structure was confirmed by uv-vis, IR and ¹H NMR spectroscopy.

### EXAMPLE 2

### Synthesis Of Octachloro Octachloro Iron (III) Chloride 1) Octachloro Zn-Complex (1h)

The octachloro Zn-complex 1h was prepared as described in Example 1.

2) Chlorination of Octachloro Zn-Complex

The zinc complex (100 mg, 0.105 mmol) was suspended in CH3OH (50 mL) and treated with NCS (140 mg, 1.05 mmol). The resulting mixture was heated at reflux for 6 h. A second portion of NCS was then added and the mixture refluxed for an additional 5 h. The mixture was then evaporated to dryness and the residue was washed with hot H2O to give the purple solid complex. The structure was confirmed by uv-vis, IR and 1H NMR spectroscopy. Demetallation of Octachloro Octachloro Zn-complex

The impure zinc complex (1.19 g; 1.25 mmol) was dissolved in CHCl₃ (300 mL), TFA (10 mL) was added and the mixture was stirred; the progress of the reaction was monitored by UV-V1S spectroscopy in CH₂Cl₂ after neutralization of the sample by Et3N. After 1 h the crude product was washed with H₂O (350 mL), aq NaHCO₃ (350 mL), H20 (2×250 mL), and dried (MgSO4). After filtration, the volume was reduced to about 100 mL and the compound was crystallized by addition of MeOH (50 mL) followed by partial evaporation of CHCl₃, affording the title complex. The structure was confirmed by uv-vis, IR and ¹H NMR spectroscopy.

3) Metal Insertion.

The free base (2.0 g; 1.715 mmol), FeCl₂-4H₂O (21.45 g; 107 mmol), and DMF (1600 mL) were degassed and refluxed under argon in a 1 L RBF. Metal insertion was completed in 16 h. Heating was discontinued and the solution was stirred open to the air and allowed to cool at room temperature. After removing solid FeCl₃ by filtration, the reaction mixture was treated with 5 N HCl 1600 mL. The hemin precipitated out as a brown solid from a clear yellow solution. After filtration, it was thoroughly washed with water and dried in a vacuum desiccator. This product was redissolved in CHCl₃ (150 mL) (the complex is significantly more soluble in chloroform than in dichloromethane) and purified by chromatography on silica (130 g; 5×17 cm). A trace of unreacted free base was eluted using CH₂Cl₂; it was followed by a trace of bluish-purple ring-opened by-product which was eluted using 2% CH₃OH in CH₂Cl₂ (v/v). The hemin was then eluted by increasing the concentration of CH₃OH to 5%. The complex was concentrated to 250 mL. To this, 5 mL conc. HCl was added and the hemin was crystallized by evaporation of CH₂Cl₂. The crystals were washed with MeOH, rinsed with pentane and dried. The structure was confirmed by uv-vis, IR and ¹H NMR spectroscopy.

### EXAMPLE 3

### Synthesis of Octachloro Octabromo Iron (III) Chloride

1) Octachloro Zn-Complex (1h)

The octachloro Zn-complex was prepared as described in Example 1.

2) Bromination of Octachloro Zn-Complex

The zinc complex 4 (2.0 g, 2.097 mmol) was dissolved/suspended in CH3OH (400 mL) and treated with Br2 (40 mL, 0.78 mol). The resulting mixture was stirred at ambient temperature for 2 hand then refrigerated overnight (4° C.). The resulting precipitate was collected and washed with a small quantity of CH3OH to furnish the green solid. The structure was confirmed by uv-vis, IR and 1H NMR spectroscopy.

### Demetallation of Octachloro Octabromo Zn-Complex

The impure zinc complex (1.19 g; 1.25 mmol) was dissolved in CHCl₃ (300 mL), TFA (10 mL) was added and the mixture was stirred; the progress of the reaction was monitored by UV-V1S spectroscopy in CH₂Cl₂ after neutralization of the sample by Et₃N. After 1 h the crude product was washed with H₂O (350 mL), aq NaHCO₃ (350 mL), H20 (2×250 mL), and dried (MgSO4). After filtration, the volume was reduced to about 100 mL and the compound was crystallized by addition of MeOH (50 mL) followed by partial evaporation of CHCl₃, affording the title complex. The structure was confirmed by uv-vis, IR and ¹H NMR spectroscopy.

### Metal Insertion.

The free base (2.0 g; 1.595 mmol), FeCl₂-4H₂O (21.45 g; 100 mmol), and DMF (1600 mL) were degassed and refluxed under argon in a 1 L RBF. Metal insertion was completed in 16 h. Heating was discontinued and the solution was stirred open to the air and allowed to cool at room temperature. After removing solid FeCl₃ by filtration, the reaction mixture was treated with 5 N HCl 1600 mL. The hemin precipitated out as a brown solid from a clear yellow solution. After filtration, it was thoroughly washed with water and dried in a vacuum desiccator. This product was redissolved in CHCl₃ (150 mL) (the complex is significantly more soluble in chloroform than in dichloromethane) and purified by chromatography on silica (130 g; 5×17 cm). A trace of unreacted free base was eluted using CH₂Cl₂; it was followed by a trace of bluish-purple ring-opened by-product which was eluted using 2% CH₃OH in CH₂Cl₂ (v/v). The hemin was then eluted by increasing the concentration of CH₃OH to 5%. The complex was concentrated to 250 mL. To this, 5 mL Conc. HCl was added and the hemin was crystallized by evaporation of CH₂Cl₂. The crystals were washed with MeOH, rinsed with pentane and dried. The structure was confirmed by uv-vis, IR and ¹H NMR spectroscopy.

### EXAMPLE 4

### Synthesis of 5,10,15,20-(4-amino)tetraphenylporphyrin

1. 5,10,15,20(4-nitro)tetra phenylporphyrin (4-nitro TPP)

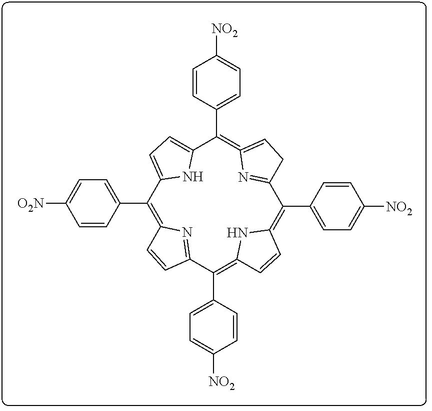

5.0 g of 4-nitrobenzaldehyde was mixed in 140 mL of propionic acid. It was shaken well. To it, freshly distilled 2.3 mL of pyrrole was added and the reaction mixture was refluxed for 45 minutes. It was cooled to room temperature and chilled to 10 to 15° C. The solid was filtered and washed with methanol and water to give 6.5 g of 5,10,15,20-(4-nitro)tetra phenylporphyrin (4-nitro TPP). Formation of 4-nitro TTP was confirmed by UV-VIS spectra (λ max=418 nm).

2. Reduction of 4-Nitro-TPP by Sodium hydrogen sulfide

5,10,15,20-(4-amino)tetraphenylporphyrin was prepared by reduction of 4-nitro-TPP using sodium hydrogen sulfide according to the following equation:

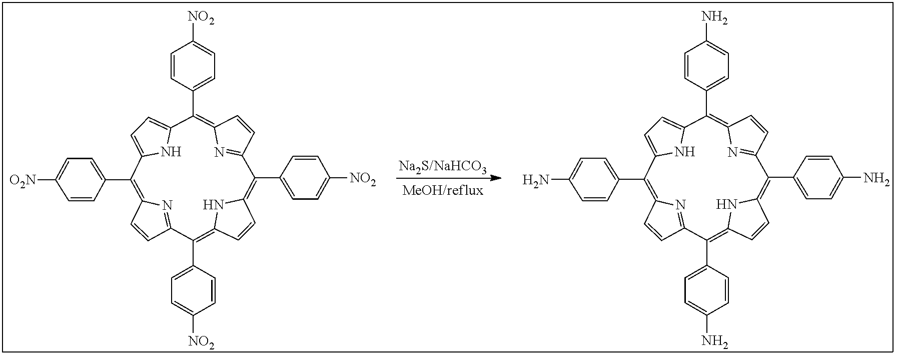 [SMILES: O=[N+]([O-])c1ccc(-c2c3nc(c(-c4ccc([N+](=O)[O-])cc4)c4ccc([nH]4)c(-c4ccc([N+](=O)[O-])cc4)c4nc(c(-c5ccc([N+](=O)[O-])cc5)c5ccc2[nH]5)C=C4)C=C3)cc1 ; Nc1ccc(-c2c3nc(c(-c4ccc(N)cc4)c4ccc([nH]4)c(-c4ccc(N)cc4)c4nc(c(-c5ccc(N)cc5)c5ccc2[nH]5)C=C4)C=C3)cc1]

Na₂S (4 g) was dissolved in 20 mL water and (4 g) of NaHCO₃ was added under stirring. When the solution became clear, 70 mL methanol was added drop wise till the complete precipitation of Na₂CO₃. It cooled and cold water (200 mL) was added to obtain 1.687 mg 5,10,15,20-(4-amino)tetraphenylporphyrin (4-amino TPP))which was filtered, washed with water and dried. Formation of 4-nitro TTP was confirmed by UV-VIS spectra (λ max=425.5 nm).

### EXAMPLE 5

### Synthesis of 5,10,15,20-(4-amino)tetraphenylporphynato Co(II) Chloride (4-Amino Co(II) TPP)

The title compound was prepared by reaction of 4-amino TPP with CoCl₂ using microwave energy according to the following reaction:

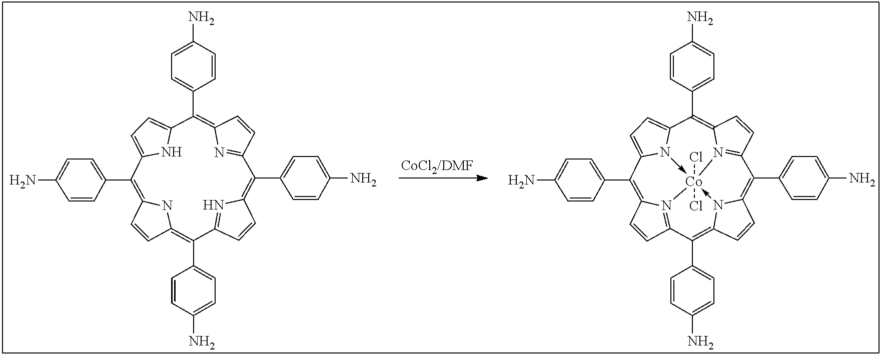

The free base 4-amino-TPP (0.050 g; 0.074 mmol), CoCl₂-6H₂O (0.017 g; 0.74 mmol), and DMF (10 mL) were irradiated in domestic microwave at low temperature. The microwave operated at 230 V at ca. 50 Hz and produced a maximum microwave power output of 750 W at a microwave frequency of 2450 MHz. The progress of the reaction was monitored by TLC. Metal insertion was completed in 20 minutes by TLC. The reaction mixture was quenched in 100 ml distilled water and the solid was separated out, filtered and dried to provide 0.065 g of the title compound. Formation of 5,10,15,20-(4-amino)tetraphenylporphynato Co(II) Chloride (4-amino Co TPP) was confirmed by UV-VIS spectra (λ438, 539.5, 583 nm).

### EXAMPLE 6

### Synthesis of 5,10,15,20-(4-amino)tetraphenylporphynato Ru(II) Chloride (4-Amino Ru (III) TPP)

The title compound was prepared by reaction of 4-amino TPP with RuCl₃ using microwave energy according to the following reaction:

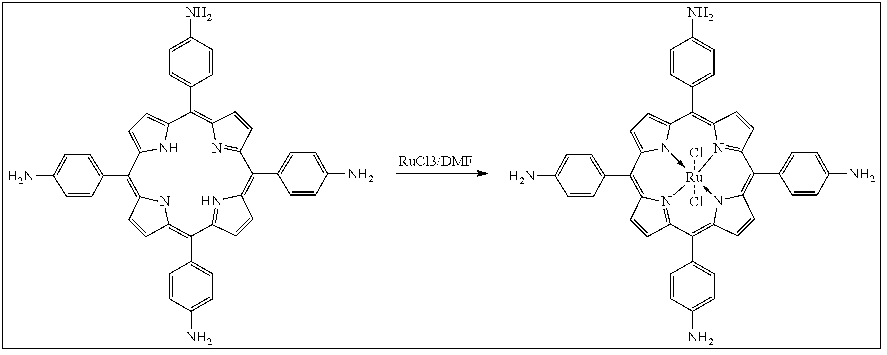

The free base 4-amino TPP (0.050 g; 0.074 mmol), RuCl₃-6H₂O (0.017 g; 0.74 mmol), and DMF (10 mL) were irradiated under domestic microwave at low temperature. The microwave operated at 230 V at ca. 50 Hz and produced a maximum microwave power output of 750 W at a microwave frequency of 2450 MHz. The progress of the reaction was monitored by TLC. Metal insertion was completed in 20 minutes. The reaction mixture was quenched in water and 100 ml and solid was separated out, filtered and dried to obtain 0.055 g of crude, 10,15,20-(4-amino)tetraphenylporphynato Ru(III) Chloride (4-amino Ru TPP). Formation of (4-amino Ru(III) TPP was confirmed by UV-VIS spectra (λ428, 517, 560, 656, 762 nm).

### EXAMPLE 7

### Synthesis of 5,10,15,20-(4-amino)tetraphenylporphynato Rh(III) Chloride (4-Amino Rh(III) TPP)

The title compound was prepared by reaction of 4-amino TPP with RhCl₃ using microwave energy according to the following reaction:

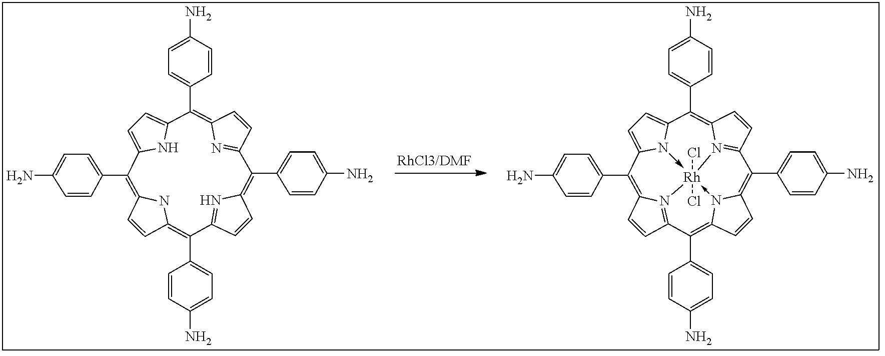

The free base 4-amino TPP (0.050 g; 0.074 mmol), RhCl3-6H2O (0.017 g; 0.74 mmol), and DMF (10 mL) were irradiated in domestic microwave at low temperature. The microwave operated at 230 V at ca. 50 Hz and produced a maximum microwave power output of 750 W at a microwave frequency of 2450 MHz. The progress of the reaction was monitored by TLC. Metal insertion was completed in 40 minutes. The reaction mixture was quenched in water and 100 ml and solid was separated out, filtered and dried to obtain 0.060 g of crude 5,10,15,20-(4-amino)tetraphenylporphynato Rh(II) Chloride (4-amino Rh TPP). Formation of 4-amino Rh TPP was confirmed by UV-VIS spectra (λ432.5, 738 nm).

### EXAMPLE 8

### Synthesis of 5,10,15,20-(4-amino)tetraphenylporphynato Fe(III) Chloride (4-Amino Fe (III) TPP)

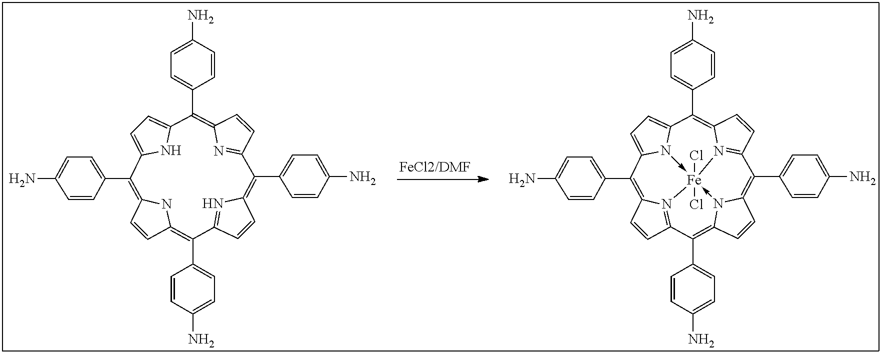

The free base Amino-TPP (0.050 g; 0.074 mmol), FeCl₂-6H₂O (0.020 g; 0.74 mmol), and DMF (10 mL) were irradiated under domestic microwave at low temperature. The microwave operated at 230 V at ca. 50 Hz and produced a maximum microwave power output of 750 W at a microwave frequency of 2450 MHz. The progress of the reaction was monitored by TLC. Metal insertion was completed in 35 minute. The reaction mixture was quenched in 100 ml distilled water and treated with 5N HCl. The solid was separated out, filter and dried. Color=Black; Wt. of compound (crude)=0.040 g

### EXAMPLE 9

### Preparation of Octachloro Octachloro Ruthenium (III) Chloride

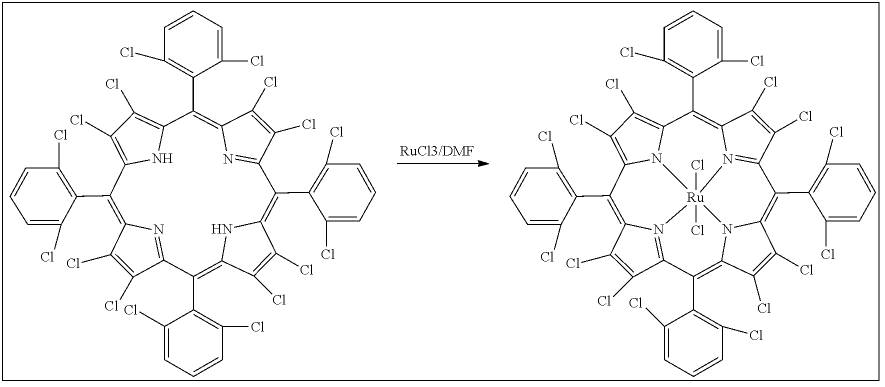

The free base Octachloro Octachloro free base (0.050 g), RuCl₃-6H₂O (0.100 g), and DMF (10 mL) were irradiated in a domestic microwave at low temperature. The microwave operated at 230 V at ca. 50 Hz and produced a maximum microwave power output of 750 W at a microwave frequency of 2450 MHz. The progress of the reaction was monitored by TLC. The irradiation was continued for 20 minutes. A small portion of reaction mixture was quenched in 10 ml water and extracted with 10 ml chloroform, to afford a purple solution. UV spectrum recorded after 20 min irradiation. Formation of 4 Octachloro Octachloro Ruthenium (III) Chloride was confirmed by UV-VIS spectra (λ ca. 420 nm). The Ru insertion occurs without destruction of the halogens on the pyrroles or the aromatic rings.

### EXAMPLE 10

### Preparation of Octachloro Octabromo Ruthenium (III) Chloride

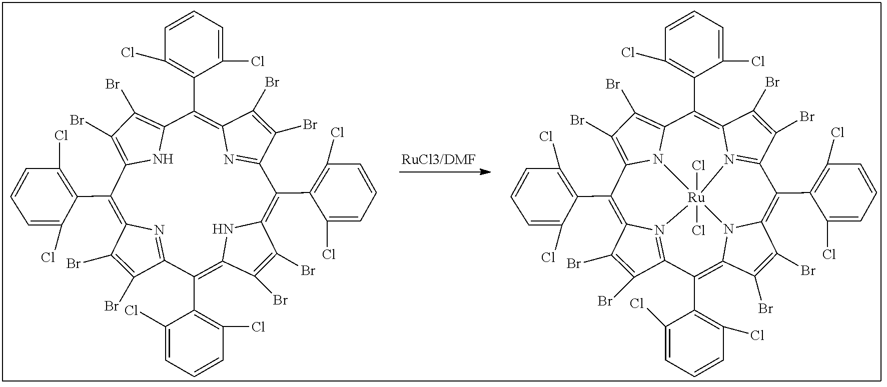

The free base Octachloro Octabromo free base (0.050 g), RuCl₃-6H₂O (0.100 g), and DMF (10 mL) were irradiated in a domestic microwave at low temperature. The microwave operated at 230 V at ca. 50 Hz and produced a maximum microwave power output of 750 W at a microwave frequency of 2450 MHz. The progress of the reaction was monitored by TLC. The irradiation was continued for 20 minutes. A small portion of reaction mixture was quenched in 10 ml water and extracted with 10 ml chloroform, to afford a yellow solution. UV spectrum recorded after 20 min irradiation. Formation of 4 Octachloro Octabromo Ruthenium (III) Chloride was confirmed by UV-VIS spectra. The Ru insertion occurs without destruction of the halogens on the pyrroles or the aromatic rings.

### EXAMPLE 11

### Preparation of 5,10,15,20-(4-amino)tetra phenylporphinato magnesium (II) Chloride

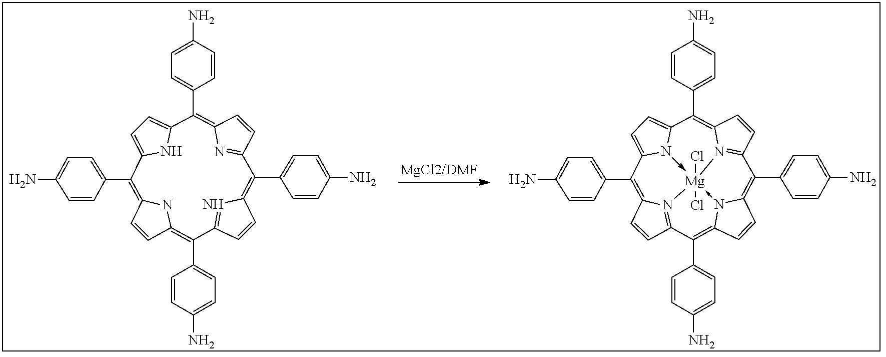

The free base 4-amino TPP (0.050 g; 0.074 mmol), MgCl₂-4H₂O (0.015 g; 0.74 mmol), and DMF (17 mL) were degassed and refluxed under argon in a RBF. The progress of the reaction was monitored by TLC. Metal insertion was completed in 3 to 5 h. Heating was discontinued and the solution was stirred open to the air and allowed to cool to room temperature. The reaction mixture was quenched in water 170 ml and stirred for 1.0 hrs. It was filtered and dried in vacuum desiccator, yielding 0.35 mg of the title compound.

### EXAMPLE 12

### Preparation of 5,10,15,20-(4-amino)tetra phenylporphinato cobalt (II) Chloride

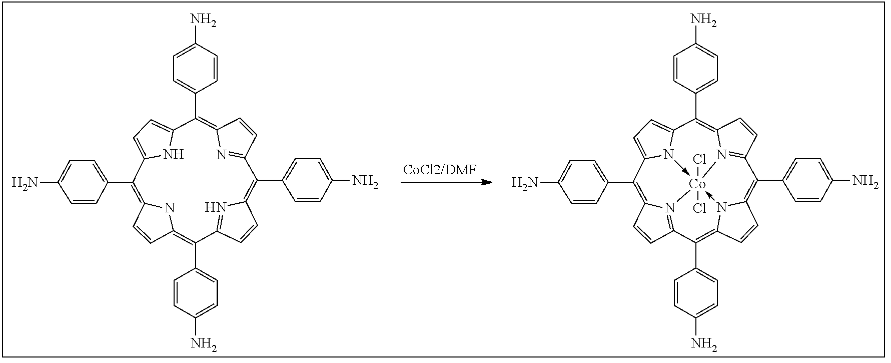

The free base 4-amino-TPP (0.050 g; 0.074 mmol), CoCl₂-6H₂O (0.017 g; 0.74 mmol), and DMF (17 mL) were degassed and refluxed under argon in a round bottom flask. The progress of the reaction was monitored by TLC. Metal insertion was completed in 3 to 5 h. Heating was discontinued and the solution was stirred open to the air and allowed to cool to room temperature. The reaction mixture was quenched in water 170 ml and stirred for 1.0 hrs. It was filtered and dried in vacuum desiccator, yielding 0.15 mg of the title compound. Formation of 4-amino Co TPP was confirmed by uv-vis spectra (λ438, 539.5, 583 nm).

### EXAMPLE 13

### Preparation of 5,10,15,20-(4-amino)tetra phenylporphinato iron (III) Chloride

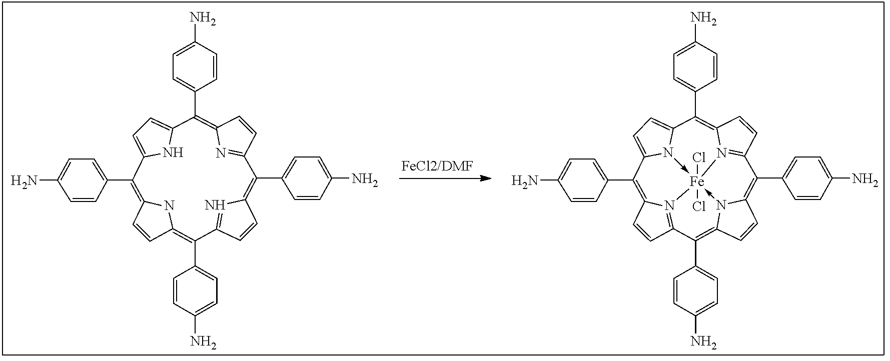

The free base Amino-TPP (0.050 g; 0.074 mmol), FeCl₂-6H₂O (0.020 g; 0.74 mmol), and DMF (17 mL) were degassed and refluxed under argon in a round bottom flask. The progress of the reaction was monitored by TLC. Metal insertion was completed in 3 to 5 h. Heating was discontinued and the solution was stirred open to the air and allowed to cool to room temperature. The reaction mixture was quenched in water 170 ml and stirred for 1.0 hrs. It was filtered and dried in vacuum desiccator, yielding 0.32 g of, 10,15,20-(4-amino)tetra phenylporphinato iron (III) (4-amino Fe (III) TPP). Formation of 4-amino Fe (III) TPP was confirmed by UV-VIS spectra (λ432 nm).

### EXAMPLE 14

### Preparation of 5,10,15,20-(4-amino)tetra phenylporphinato nickel (II) Chloride

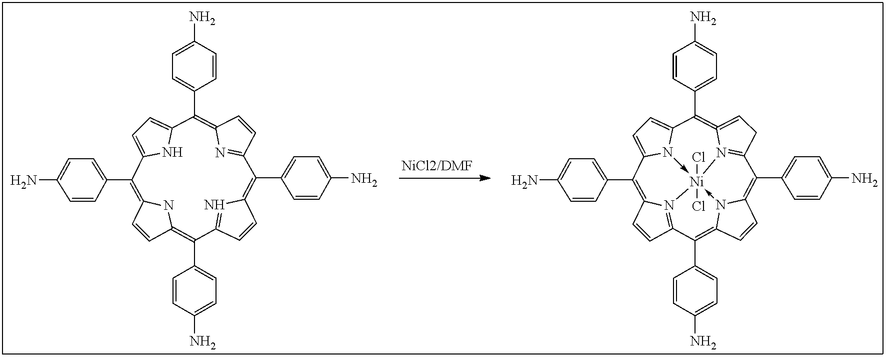

The free base 4-amino TPP (0.050 g; 0.074 mmol), NiCl₂-6H₂O (0.017 g; 0.74 mmol), and DMF (17 mL) were degassed and refluxed under argon in a RBF. The progress of the reaction was monitored by TLC. Metal insertion was completed in 3 to 5 h. Heating was discontinued and the solution was stirred open to the air and allowed to cool to room temperature. Formation of 5,10,15,20-(4-amino)tetra phenylporphinato nickel (II) (4-amino Ni (II) TPP) was confirmed by UV-VIS spectra (λ437.5, 722 nm).

### EXAMPLE 15

### Preparation of polystyrene supported chloro [meso-(2,6-dichlorophenyl)porphinato] Iron (III)

Polystyrene (5 g) was dissolved in 50 ml of CH₂Cl₂at 40° C. Chloro [meso-(2,6-dichlorophenyl)porphinato) Iron (III) (0.5 g) was added and the dark colored solution was stirred for 1 hour. Cooling of the solution to 0° C. and further addition of 60 ml of ethanol (drop by drop) separates out a thick, highly viscous mass, which on drying gave a polystyrene supported catalyst. Weight of the encapsulated catalyst: 5.36 g (97.82%).

### EXAMPLE 16

### Preparation of polystyrene supported [meso-tetra phenyl porphinato] cobalt (II)

Polystyrene (5 g) was dissolved in 50 ml of CHCl₃ at 50° C. [Meso-tetra Phenyl porphinato] cobalt (II)(0.5 g) was added and the dark colored solution was stirred for 1 hour. Cooling of the solution to 0° C. and further addition of 50 ml of methanol (drop by drop) separates out a thick, highly viscous mass, which on drying gave a solid catalyst [meso-tetra phenyl porphinato] cobalt (II). Weight of the encapsulated catalyst: 5.42 g (98 54).

### EXAMPLE 17

### Preparation of polystyrene supported 5,10,15,20-(4-nitro)tetra phenylporphinato nickel (II)

Polystyrene (5 g) was dissolved in 50 ml of CH₃ at 50° C. 5,10,15,20-(4-nitro)tetra phenylporphinato nickel (II)(0.5 g) was added and the dark colored solution was stirred for 1.5 hour. Cooling of the solution to 0° C. and further addition of 50 ml of methanol (drop by drop) separates out a thick, highly viscous mass, which on drying gave a solid catalyst 5,10,15,20-(4-nitro)tetra phenyl porphinato nickel (II). Weight of the encapsulated catalyst: 5.42 g (98.6%).

### EXAMPLE 18

### Extraction of Lignin from a Biomass Source

The example reports extraction of Custard Apple plant leaves, however, the process can be employed for extraction of any biomass. Custard Apple plant leaves were dried in room temperature (7 days not in direct sun light), ground as a fine powder.

Extraction follows sequentially in the order of

1. Water

2. Ethanol

3. Chloroform

4. Acetone

5. Acidic Dioxane (Nitrogen atmosphere)

### 1. Water Extraction:

The Custard Apple plant leaves powder was filled in the 100 ml Soxhlet extractor apparatus (thimbles). The extraction of leaves was continued until no color leached (light brown) from the walls, (near about 42 hr. in the time interval of 8 hours/day, it took 5 days for the complete extraction of the water component) of Soxhlet apparatus. Extracted wood was dried in room temperature for 2 day. The extracted water component was filtered and filtrated was distilled off on a heating mantle and stored in the refrigerator, for the further analysis.

### 2. Ethanol Extraction:

The extracted Custard Apple plant leaves were extracted with ethanol until no color leached from the wall of the Soxhlet apparatus (it took 3 days in the time interval of 8 hours/day). The extracted ethanol component was filtered and filtrated was distilled off on a heating mantle and stored in the refrigerator, for the further analysis. The extracted leaves where dried.

### 3. Chloroform Extraction

The extracted leaves were again filled into the Soxhlet extractor and extraction continuous until no color leach by chloroform (extraction was continued for 2 days, accordingly 8 hours/day). The extracted leaves where dried and use for further extraction.

### 4. Acetone Extraction.

The extracted Custard Apple plant leaves powder was filled in the 100 ml Soxhlet extractor apparatus (thimbles). The extraction of leaves was continued till the no color leached (light brown) from the walls, of Soxhlet apparatus (extraction was completed in 2 days, in the time interval of 8 hours/day). Extracted wood was dried in room temperature. The extracted acetone component was filtered and filtrated was distilled off on a heating mantle and stored in the refrigerator, for the further analysis. The extracted leaves where dried and use for further process.

### 5. Acidic Dioxane Extraction.

The dry cell wall material was placed in a round bottom flask and into this acidic dioxane (90 mL dioxane+10 mL 2N HCl solution) was added; the flask was connected to the refluxed condenser and N₂ gas was blown onto the liquid surface for 20-30s. The solution was then refluxed under nitrogen for 45 min. After cooling the solution was filtered through a glass fiber filter (GF/C, 47 mm, Whatman) paper and collected in Erlenmeyer flask, 96% dioxane was used to wash the residue collected on the filter and the wash was combined with the original filtrate. Sodium carbonate was added to the Erlenmeyer flask and the sample placed on stirring for several minutes until neutralization of the solution (measured with a pH strip).

The solution was filtered through a 0.45 μm nylon membrane before concentrating to 10-15 mL, under reduced pressure on a rotary evaporator. The solution was added drop wise into rapidly stirring distilled water. Any insoluble residue remaining in the flask was washed with 96% dioxane and added drop wise to the water. Into this sodium sulfate was added for flocculation of sample.

After stirring the precipitate was pelleted by centrifugation and supernatant was removed. Lignin residues were dissolved in dioxane, filtered through a 0.45 μm nylon membrane, and added drop wise to rapidly stirring anhydrous diethyl ether. The resulting precipitate was pelleted by centrifuging and the entire solubilization in dioxane and other wash step was repeated to remove hydrophobic nonlignin contaminants. After removing the diethyl ether, petroleum ether was added while stirring to thoroughly wash the lignin residue. This solvent was removed after allowing the residue to settle. The lignin residue was freeze-dried for 48 h and stored.

Cell wall material used=67 g

Wt. of lignin=0.586 g

### EXAMPLE 19

### Depolymerization Of Lignin By Iron (III) Tetraphenyl Porphyrin (Fe TPP)

To a solution of lignin (50 mg) (purified as described in Example 4) in ethyl acetate and water (1:1) 20 mL, TPP-iron complex (5 mg) was added, which was stirred for 5 min under room temperature. Into the mixture (6 mL) sodium hypochlorite was added within 10 min and stirred at room temperature for 30 min. The reaction was monitored by thin layer chromatography (TLC), to this reaction mixture NaCl was added and the reaction mixture was extracted with ethyl acetate, the organic layer was dried.

TLC (EtOAc:Hexane (1:9)) was used to monitor the progress of the depolymerization reaction to the degradation products that are typical of cellulose and hemicellulose. TPP was not a strong catalyst and the consumption of TPP was complete after a 3-5 minutes, as indicated by the disappearance of the spot on the TLC plate attributed to lignin. It is estimated that TPP had a turnover of 5-10.

| Spot | |
|---|---|
| (Compound) | Rf Values |
| Lignin | 0.0 |
| Spot 1 | 0.21 |
| Spot 2 | 0.42 |
| Spot 3 | 0.57 |
| Spot 4 | 0.68 |
| Spot 5 | 0.76 |
| Spot 6 | 0.89 |

### Destructive Oxidation of Polymers

### EXAMPLE 20

### Depolymerization of Lignin by Octachloro-Iron (III) Tetraphenyl Porphyrin (Octachloro Fe TPP)

Lignin was obtained using extraction methods substantially as described by Romualdo S. Fukushima and Ronald D. Hatfield, Journal of agricultural and food chemistry, July 2001, Vol. 49, Number 7, pp. 3133-3139.

To the solution of lignin (50 mg) in ethyl acetate and water (1:1) 20 mL, Octachloro-iron (III) TPP complex (5 mg) was added, which was stirred for 5 min under room temperature. Into the mixture (6 mL) sodium hypochlorite was added within 10 min and stirred at room temperature for 30 min. The reaction was monitored by TLC, to this reaction mixture NaCl was added and the reaction mixture was extracted with ethyl acetate, the organic layer was dried.

TLC (EtOAc:Hexane (1:9)) was used to monitor the progress of the depolymerization reaction to the degradation products that are typical of cellulose and hemicellulose. Octachloro-Iron (III) TPP was a strong catalyst and monitoring by TLC indicated that Octachloro-iron (III) TPP was still present and chemically active after several hours. Octachloro-Iron (III) TPP keeps functioning for hours for a catalytic turnover of at least 10,000.

| Spot (Compound) | Rf Values |
|---|---|
| Lignin | 0.0 |
| Spot 1 | 0.47 |
| Spot 2 | 0.68 |
| Spot 3 | 0.85 |
| Spot 4 | 0.98 |

### EXAMPLE 21

### Comparative Study of Depolymerization of Sugar Cane by Octa-Chloro TPP (OC), Octachloro-Octachloro TPP (OCOC) and Octachloro-Octabromo-Iron (III) TPP (OCOB)

To a solution of selected solvent (25 mL), and iron complexes (5 mg), dried sugar cane (1 g) was added and the mixture was stirred for 1 h. To that 6 mL sodium hypochlorite was added, the resulting suspension was stirred for 55 h at room temperature reaction was monitored by TLC. Then the mixture was filtered and the solvents were removed under reduced pressure. Unlike TPP, which survives only for a few minutes, all three activated catalysts demonstrated catalytic activity for several hours. Both Octachloro-Octachloro-Iron (III) TPP and Octachloro-Octabromo-Iron (III) TPP continued oxidizing for 55 hours and more. The Comparative Table below summarizes the reaction conditions and TLC of the tested mixtures.

COMPARATIVE TABLE

| | Wt. of | | Solvents (mL) | | | | | Rf Values | | |
|---|---|---|---|---|---|---|---|---|---|---|
| Metal | metal | Wt. of | | | 10% | | TLC | | | 10% |
| complexes | complexes | Sugarcane | | | MeOH | NaOCl | (EtOAc:Hexane | | | MeOH |
| of iron (III) | (mg) | (gm) | MeOH | IPA | in IPA | (mL) | Ratio) | MeOH | IPA | in IPA |
| OC | 5.0 | 1.0 | 25 | 25 | 25 | 6.0 | 1.5:8.5 | 0.07, 0.12, | 0.34, 0.56 | 1.39, |
| | | | | | | | | 0.17, 0.24, | | 0.56, |
| | | | | | | | | 0.39, 0.49 | | 0.80 |
| OCOC | 5.0 | 1.0 | 25 | 25 | 25 | 6.0 | 1.5:8.5 | 0.10, 0.22, | 0.15, 0.32, | 0.46, |
| | | | | | | | | 0.36, 0.46, | 0.49, 0.56, | 0.66, |
| | | | | | | | | 0.61, 0.80 | 0.83 | 0.80 |
| OCOB | 5.0 | 1.0 | 25 | 25 | 25 | 6.0 | 1.5:8.5 | 0.07, 0.10, | 0.32, 0.44, | 0.41, |
| | | | | | | | | 0.17, 0.27, | 0.61 | 0.59 |
| | | | | | | | | 0.37, 0.41 | | |

After TLC indicated that all lignin in the mixture had been depolymerized, a fresh batch of biomass was introduced into the reaction vessels containing Octachloro-Octachloro-Iron (III) TPP and Octachloro-Octabromo-Iron (III) TPP. Upon addition of new biomass, the catalyst began to depolymerize lignin, indicating that the catalyst was still active.

### EXAMPLE 22

### Depolymerization of Various Biomass Sources with Cane Sugar by Octa-Chloro, Octachloro-Octachloro and Octachloro-Octabromo-Iron (III) TPP to Obtain Phenols

The depolymerization of lignin from grass, neem leaves, banana leaves and coconut fibers was investigated using the following procedure. To a selected solvent, e.g., ethyl acetate and toluene (1:1) 100 mL and iron complexes (50 mg) dried biomass (5 g) were added. 10 mL sodium hypochlorite was added and the resulting suspension was heated for 55 h at 60-66° C.

In addition, purified lignin obtained by extraction from each of these sources (using extraction methods substantially as described by Romualdo S. Fukushima and Ronald D. Hatfield, Journal of agricultural and food chemistry, July 2001, Vol. 49, Number 7, pp. 3133-3139)) also was investigated using the following procedure. In a 50 mL beaker, 50 mg lignin was placed; into that 25 mL ethyl acetate and toluene (1:1) was added and the reaction mixture was warmed to dissolve the lignin. The mixture was then cooled and stirred for 5 min. To that stirred mixture was added iron complexes (10 mg), followed by sodium hypochlorite (2 mL). The mixture was stirred for 30 minutes.

TLC comparison (ethyl acetate:n-hexane (1.5:8.5) was carried out for the depolymerized materials from both lignin and biomass sources. The lignin is completely depolymerized; the catalyst remains stable and active. Dilution of the aqueous organic reaction mixture with organic solvent followed by back extraction of the reaction mixture with sodium hydroxide or aqueous alkali furnished the requisite phenols.

The presence of available cellulose was verified by reaction of the delignified biomass material with cellulose enzyme and determination of the presence of glucose, a reaction product of cellulose digestion with enzyme. The cellulose enzyme is NKL biozyme (from Bangalore, India) which is supplied as powder and contains 12,000 units of xylanase and 20,000 units of cellulase per gram. The de-lignified biomass was boiled for 1 min. to clear bacterial contamination and was then cooled to room temperature and filtered. A 10 mL aliquot was combined with a 10 mL aliquot of an 30 wt % suspension of cellulose in PBS solution (75 g/150 mL PBS) and the mixture was incubated at 42° C. for up to 24 h. The powder was added to the mix after delignification to a final concentration of 300 units per gram of dry mass.

The mixture was tested for the presence of glucose using the protocol described in A. F. Mohum and I. J. Y. Cook, J. Clin. Path. (1962), 15, 169-180). Aliquots were taken after 6 h, 10 h and 24 h of incubation and were tested for the presence of glucose. The color change (which deepened in intensity with the incubation time) indicated that glucose formation was achieved by enzymatic action on the depolymerized biomass, as compared to a control.

### Destructive Oxidation of Organic Dyes

The decomposition of the following commercial dyes were observed visually and by uv-vis spectroscopy.

Black dye: (azo)-5-amino-4-hydroxy-di(hydrogen sulfate)ester, tetrasodium salt;2,7-napthalenedisulficicacid, 3,6-(bis(4-((2-hydroxyethyl)sulfonyl)phenyl)bis; 2,7-napthalenedisulfonicacid, 4-amino-5-hydroxy-3,6-bis[[4-[[2-(sulfooxy)ethyl; CAS 17095-24-8

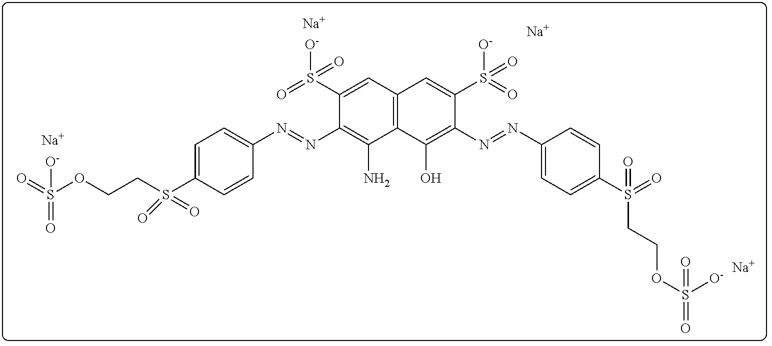 [SMILES: [Na+] ; [Na+] ; Nc1c(/N=N/c2ccc(S(=O)(=O)CCOS(=O)(=O)[O-])cc2)c(S(=O)(=O)[O-])cc2cc(S(=O)(=O)[O-])c(/N=N/c3ccc(S(=O)(=O)CCOS(=O)(=O)[O-])cc3)c(O)c12 ; [Na+] ; [Na+]]

Red Dye: Congo Red (sodium sodium 3,3′-([1,1′-biphenyl]-4,4′-diyl)bis(4-aminonaphthalene-1-sulfonate))

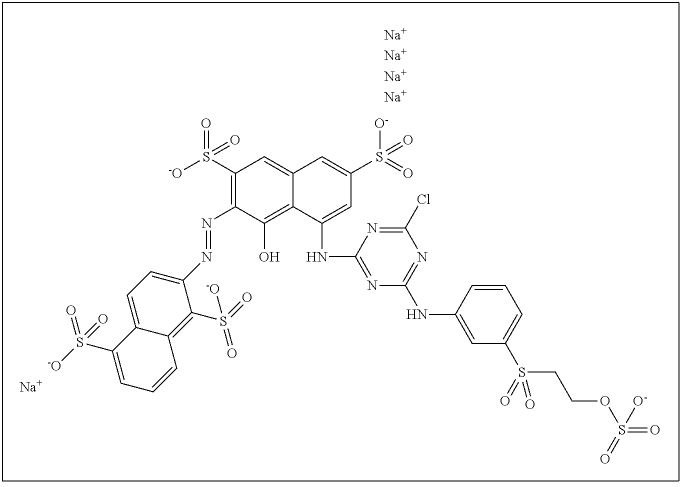

Yellow Dye: Dipotassium 4-[4-[2,5-dimethoxy-4-(2-sulfonatooxyethylsulfonyl)phenyl]diazenyl-3-methyl-5-oxo-4H-pyrazol-1-yl]benzensulfonate; CAS 20317-19-5

Blue Dye: Structure not report

### EXAMPLE 23

### Destructive Oxidation of Organic Dyes Using meso-tetrakis(2,6-dichlorophenyl)β-octabromo porphinato iron (III) chloride [Octachloro Octabromo Iron (III) Chloride]

Standard commercial yellow, blue, red and black dyes were treated with meso-tetrakis(2,6-dichlorophenyl)β-octabromo porphinato iron (III) chloride [Octachloro Octabromo Iron (III) Chloride] and sodium hypochlorite as the co-oxidizer and demonstrated rapid decomposition of the organic dyes into hydroxylated, epoxylated and oxidized compounds.

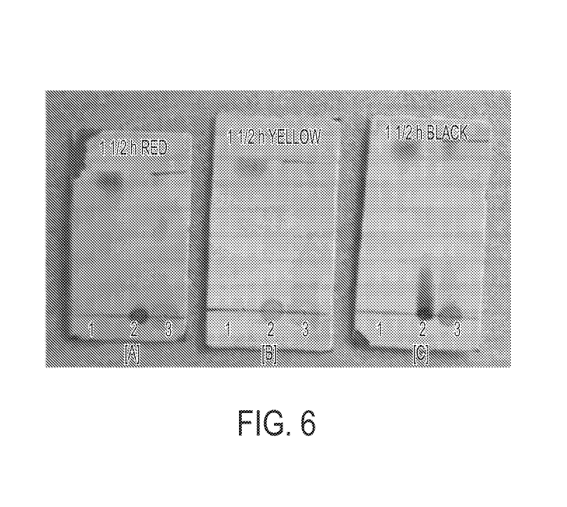

In a 250 mL conical flask 1 gm of dyes was taken; to that 100 ml distilled water was added. The reaction mixture was heated under stirring when temperature reaches 40-50° C., 25 mg OCOB Fe³⁺ TPP in 10 mL ethyl acetate was added along with 5 mL sodium hypochlorite. The reaction was monitored by TLC at different time intervals (15 min, 30 min, 60 min, and 90 min). FIG. 5 is a TLC plate 15 minutes after treatment of red dye (A), yellow dye (B) and black dye (C) with Octachloro Octabromo Fe TPP illustrating the decomposition of the dye. The first channel on each plate is Octachloro Octabromo Fe TPP, the second channel is untreated dye and the third channel is the treated dye after 15 minutes. Note the dramatic decrease in color after only 15 minute. Even more dramatic color loss is noted after 1.3 hours, as is shown in the TLC plates in FIG. 6.

### EXAMPLE 24

### Destructive Oxidation of Organic Dyes Using Modified Jacobsen's Catalyst

Standard commercial red, yellow, blue and black dyes were treated with Modified Jacobsen Catalyst and sodium hypochlorite as the co-oxidizer and demonstrated rapid decomposition of the organic dyes into hydroxylated, epoxydized, dihydroxylated, and oxidized compounds.

In a 250 mL conical flask 1 gm of dye was taken; to that 100 ml distilled water was added. A 20 mL aliquot was added to a round bottom flask and heated to a boil. 10 mL solubilized Modified Jacobsen's Catalyst (10 mg) in ethyl acetate 10 mL was added drop wise under stirring. To the boiled reaction mixture sodium hypochlorite (10 mL) was added; the reaction was monitored at different time intervals. Before the addition of sodium hypochlorite the color of the dye remains unchanged, even at reflux temperatures. After the addition of sodium hypochlorite drastic changes were observed as reported below. The reaction is very rapid indicating that complete oxidation of the dye has taken place. The proposed chemistry that is likely occurring is primarily oxidation, hydroxylation and epoxidation. We refluxed for an additional two hours to effect oxidation at all available sites in the molecule.

Firstly, two layers were observed after instant addition of sodium hypochlorite. Secondly, with the continuous heating the maximum clear solution was achieved after half hour. It remained so through the heating and also after the reaction mixture was cooled to room temperature.

Results are summarized in Table 1.

| | Time | | | |
|---|---|---|---|---|
| | Before | Immediately on | | |
| | addition | addition of | | |
| Dye | of oxidizer | oxidizer | ½ hour reflux | 2 hour reflux |
| RED | Deep red | Two phases, one | Single phase; | Single phase; |
| | solution | deep red and | pale red | almost |
| | | other pale red | | completely |
| | | | | decolorized |
| BLUE | Deep blue | Two phases, one | Single phase; | Single phase; |
| | solution | deep brown and | pale red | almost |
| | | other pale brown | | completely |
| | | | | decolorized |
| YELLOW | Deep | Two phases, one | Single phase; | Single phase; |
| | yellow | deep red and | pale red | almost |
| | solution | other pale red | | completely |
| | | | | decolorized |
| BLACK | Deep | Two phases, one | Single phase; | Single phase; |
| | black | deep red and | pale red | almost |
| | solution | other pale red | | completely |
| | | | | decolorized |

### EXAMPLE 25

Standard commercial red, blue and black dyes were treated with Modified Jacobsen Mn catalyst

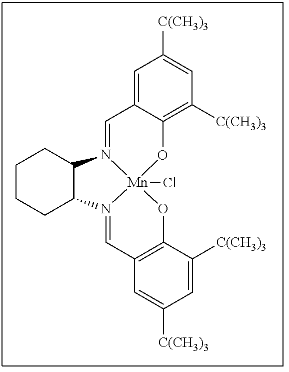

and sodium hypochlorite as the co-oxidizer substantially as described herein above. The compositions demonstrated rapid decomposition of the organic dyes into hydroxylated, epoxylated and oxidized compounds, as demonstrated by uv-vis monitoring.

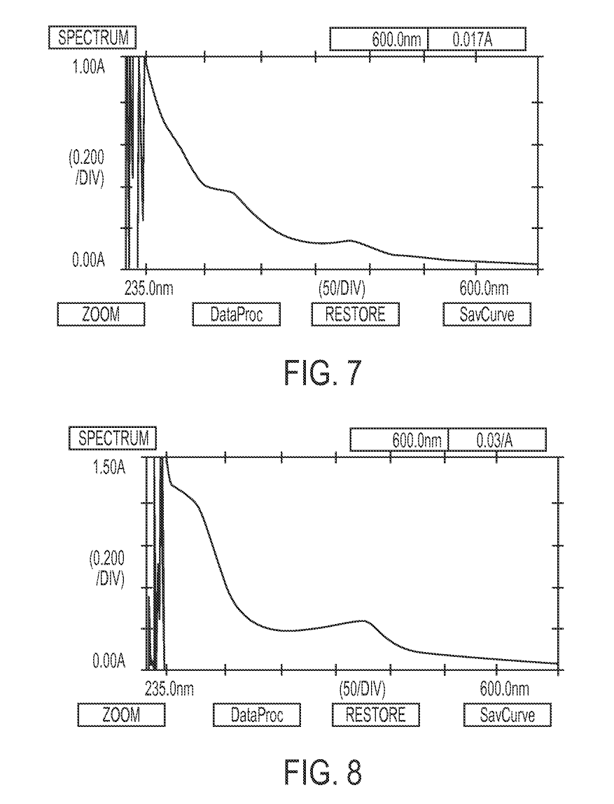

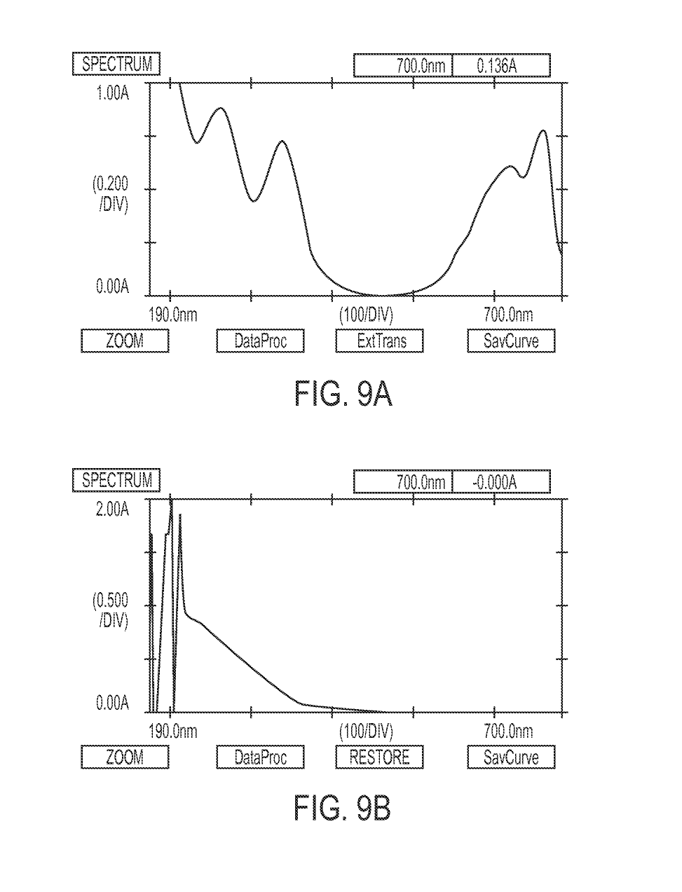

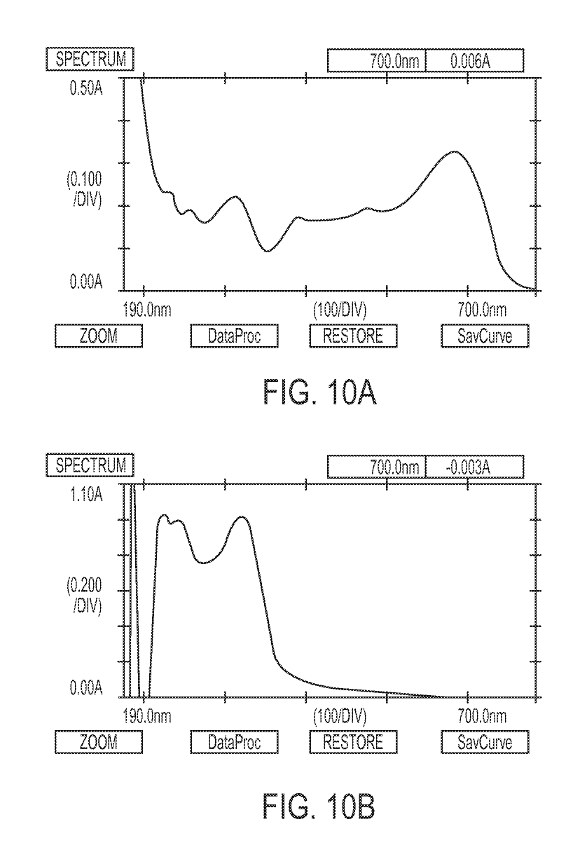

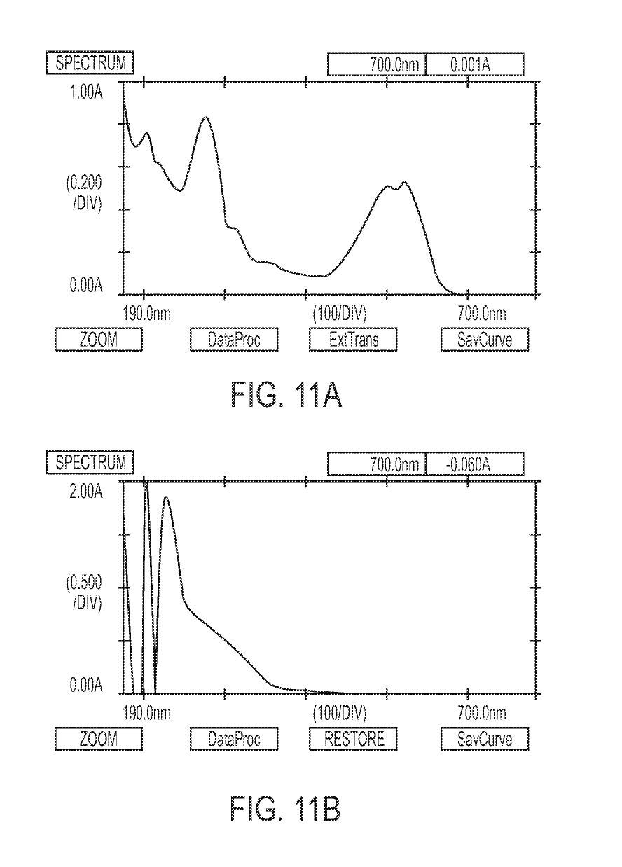

FIG. 7 shows the uv-visible spectrum for the Modified Jacobsen catalyst, illustrating a weakly absorbing peak at 412 nm. FIG. 9A shows the uv-visible spectrum for the blue dye exhibiting strong absorption in the visible energy region with peaks at about 668 nm and 627 nm. FIG. 9B is a uv-visible spectrum of the blue dye after treatment with Jacobsen's Mn catalyst and sodium hypochlorite after ½ hour showing absence of absorption in the visible region of the spectrum. Similar spectra are observed on testing up to two hours after exposure. FIG. 10A shows the uv-visible spectrum for the black dye exhibiting strong absorption in the visible energy region with peaks at about 597 nm, 484 nm and 396 nm. FIG. 10B is a uv-visible spectrum of the black dye after treatment with Modified Jacobsen Modified Jacobsen's Mn catalyst and sodium hypochlorite after ½ hour showing absence of absorption in the visible region of the spectrum. Similar spectra are observed on testing up to two hours after exposure. FIG. 11A shows the uv-visible spectrum for the red dye exhibiting strong absorption in the visible energy region with peaks at about 543 nm and 520 nm, FIG. 11B is a uv-visible spectrum of the red dye after treatment with Modified Jacobsen Modified Jacobsen's Mn catalyst and sodium hypochlorite after ½ hour showing absence of absorption in the visible region of the spectrum. Similar spectra are observed on testing up to two hours after exposure.

### EXAMPLE 26

The above example is repeated, except that Modified Jacobsen Co Catalyst is used. As with Modified Jacobsen Mn catalyst, absorption in the visible region of the spectrum disappears after exposure to the oxidation catalyst for all three dyes.

### EXAMPLE 27

The above example is repeated, except that OCOBFe³⁺ complex is used as the catalyst. As with Modified Jacobsen Mn catalyst, absorption in the visible region of the spectrum disappears after exposure to the oxidation catalyst for all three dyes.

It will be appreciated that while a particular sequence of steps has been shown and described for purposes of explanation, the sequence may be varied in certain respects, or the steps may be combined, while still obtaining the desired configuration. Additionally, modifications to the disclosed embodiment and the invention as claimed are possible and within the scope of this disclosed invention.

What is claimed is:

1. A method of decomposing an organic substrate comprising:
identifying an organic substrate having one or more undesired properties; and
contacting the organic substrate with an oxidizing agent and a catalyst selected from the group consisting of sterically hindered and electronically activated metallotetraphenylporphyrins, metallophthalocyanines and metallosalen complexes in an aqueous solution to produce a treated composition comprising one or more degradation products, wherein the degradation products have one or more desired properties and/or lack the undesired properties of the organic substrate.

2. The method of claim 1, wherein the organic substrate is toxic and the degradation products are less toxic than the organic substrate.

3. The method of claim 1, wherein the organic substrate is an organic dye.

4. The method of claim 3, wherein the degradation products are colorless.

5. The method of claim 1, wherein the degradation products have increased water solubility relative to the organic substrate.

6. The method of claim 1, wherein the organic substrate is a polymer and the degradation polymer is one or more of monomers or oligomers.

7. The method of claim 6 wherein the polymer comprises lignin.

8. The method of claim 6 wherein the polymer comprises plastics.

9. The method of claim 6, wherein the polymer is selected from the group consisting of polyethylenes, polypropylenes, polystyrenes, polyurethanes, and polyalkoxy polymers and mixtures thereof.

10. The method of claim 1, wherein the catalyst is a meso-tetraphenyl porphyrin.

11. The method of claim 1, wherein the catalyst is phthalocyanine.

12. The method of claim 10, wherein the meso-tetraphenylporphyrin catalyst comprises at least one halide substitution on the phenyl groups of meso-tetraphenylporphyrins or on the β-pyrrolic positions of the porphyrin.

13. The method of claim 11, wherein the phthalocyanine comprises at least one halide substitution on the benzo groups of the phthalocyanine

14. The method of claim 1, wherein the catalyst is a compound 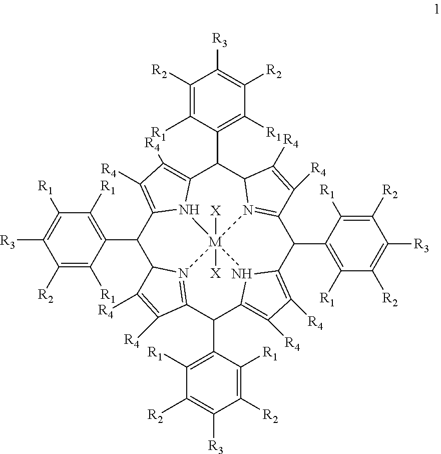
wherein R¹ is the same or different and is selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,
R², R³ or R⁴ are the same or different and are selected from the group consisting of H, Cl, Br, CH₃, SO₃⁻, CN, [N(R′)₃]^{|}, COOR′, —OCONR′₂, —OMOM, CON—R′, CONR′₂, CH═NR′, SO₂NR′₂, SO₂R, CF and NO₂,
R′ is H or a C1-C6 alkyl,
M is a transition metal, such as Fe, Zn, Co, Ni, Cu, Mn, Rh, Mg, Ru, Pt, and Pd., and
and optionally wherein one or more axial ligands X selected from the group halogens (F, Cl, Br), OH, OCl, CO, [N(R′)₃]⁺, substituted or unsubstituted pyrimidine or imidazole bases is included and/or a counter ion is included to maintain charge neutrality.

15. The method of claim 1, wherein the catalyst is a compound 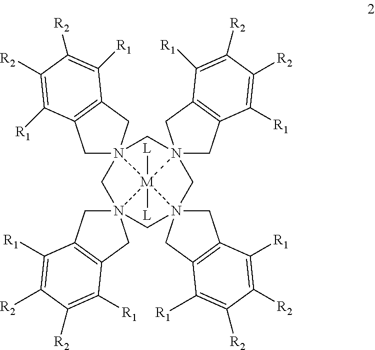
wherein R¹ is the same or different and is selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,
R² is the same or different and is selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,
wherein R′ is H or a C1-C6 alkyl,
M is a transition metal, such as Fe, Zn, Co, Ni, Cu, Mn, Rh, Mg, Ru, Pt, and Pd,
and optionally wherein one or more axial ligands X selected from the group halogens (F, Cl, Br), OH, OCl, CO, [N(R′)₃]⁺, substituted or unsubstituted pyrimidine or imidazole bases is included and/or a counter ion is included to maintain charge neutrality.

16. The method of claim 1, wherein the catalyst is a compound 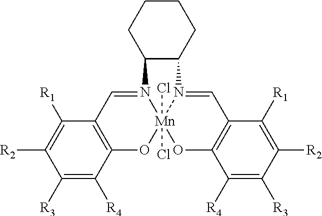
wherein R¹ is the same or different and is selected from the group consisting of Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,
R² is the same or different and is selected from the group consisting of H, Cl, Br, CH₃, SO₃⁻, CN, [N(R′)₃]⁺, COOR′, —OCONR′₂, —OMOM, CON—R′, CONR′₂, CH═NR′, SO₂NR′₂, SO₂R, CF and NO₂,
wherein R′ is H or a C1-C6 alkyl,
M is a transition metal, such as Fe, Zn, Co, Ni, Cu, Mn, Rh, Mg, Ru, Pt, and Pd,
and optionally wherein one or more axial ligands X selected from the group halogens (F, Cl, Br), OH, OCl, CO, [N(R′)₃]⁺, substituted or unsubstituted pyrimidine or imidazole bases is included and/or a counter ion is included to maintain charge neutrality.

17. The method of claim 1, wherein the catalyst is present at less than 5% wt/wt catalyst/organic substrate.

18. The method of claim 1, wherein the catalyst is present at less than 1% wt/wt catalyst/organic substrate.

19. The method of claim 1, wherein the catalyst is present at less than 0.5% wt/wt catalyst/organic substrate.

20. The method of claim 1, wherein the catalyst is a homogenous catalyst.

21. The method of claim 1, wherein the catalyst is a heterogeneous catalyst.

22. A method for obtaining phenol, comprising:
contacting a lignin-containing composition with a oxidizing agent and a catalyst in an aqueous solution to produce a treated composition containing phenol, wherein the catalyst is selected from the group consisting of sterically hindered and electronically activated metallotetraphenylporphyrins, metallophthalocyanines and metallosalen complexes.

23. The method of claim 22, wherein the biomass comprises plant material.

24. The method of claim 22, wherein the biomass is obtained from perennial woody plants, graminoids, herbaceous plants, monocots, and dicots.

25. The method of claim 22, wherein the biomass comprises by -products and waste from livestock farming, food processing and preparation and domestic organic waste.

26. The method of claim 22, wherein biomass is selected from the group consisting of s bagasse, leafy or woody biomass, sugar cane, grass, neem, eucalyptus, wood (saw dust, wood chips, bark, etc.), sawdust or wood chips from foresting operations, neem plant residues from the processing of neem plant oil.

27. The method of claim 22, wherein oxidizing agent is selected from the group consisting of organic and inorganic peroxides, oxygen donor molecules, peracids, hypochlorites, ozone, potassium hydrogen persulfate, 2,6-dichloropyridine-N-oxide and molecular oxygen.

28. The method of claim 22, wherein the reaction is conducted in an aqueous system.

29. The method of claim 22, wherein the process is a continuous process.
